(19) United States
(12) Patent Application Publication
(10) Pub. No.: US 2018/0016548 A1
(43) Pub. Date: Jan. 18, 2018

(54) ACELLULAR SCAFFOLDS FOR MATURATION OF IPSC-HEPATOCYTES

(71) Applicant: Northwestern University, Evanston, IL (US)

(72) Inventors: Jason A. Wertheim, Evanston, IL (US); Bo Wang, Evanston, IL (US)

(21) Appl. No.: 15/650,481

(22) Filed: Jul. 14, 2017

Related U.S. Application Data
(60) Provisional application No. 62/362,437, filed on Jul. 14, 2016.

(51) Int. Cl. C12N 5/071; C12N 5/077

(52) U.S. Cl. CPC C12N 5/067; C12N 5/0652; C12N 2523/00

(57) ABSTRACT

Provided herein are compositions and systems comprising induced pluripotent stem cell (iPSC)-derived hepatocytes and systems and methods for acellular maturation thereof. In particular, iPSC-derived hepatocytes are matured on decellularized extracellular matrix (ECM) to yield cells with enhanced hepatocytic biomarkers and functionality.

### CROSS-REFERENCE TO RELATED APPLICATION

The present application claims priority to U.S. Provisional Patent Application Ser. No. 62/362,437 filed Jul. 14, 2016, which is hereby incorporated by reference in its entirety.

### STATEMENT REGARDING FEDERAL FUNDING

This invention was made with government support under K08 DK101757 awarded by the National Institutes of Health. The government has certain rights in the invention.

### FIELD

Provided herein are compositions and systems comprising induced pluripotent stem cell (iPSC)-derived hepatocytes and systems and methods for acellular maturation thereof. In particular, iPSC-derived hepatocytes are matured on decellularized extracellular matrix (ECM) to yield cells with enhanced hepatocytic biomarkers and functionality.

### BACKGROUND

Induced pluripotent stem cell (iPSC) technology is a powerful strategy to develop multipotent cells from mature tissues with the ability to direct differentiation toward any tissue lineage to model disease (refs. 1-6; incorporated by reference in their entireties), analyze drug pharmacokinetics (ref 7; incorporated by reference in its entirety), and potentially develop cells as therapies for a variety of diseases (refs. 8-11; incorporated by reference in their entireties). The development of ‘patient specific’ hepatocytes with intact synthetic xenobiotic catalytic activity would accelerate pharmaceutical testing and drug development, yet the ability of iPSC-derived hepatocytes to replicate the function of endogenous cells is limited by a blunted phenotype with reduced synthetic ability and metabolic enzyme activity.

Primary hepatocytes are in high demand as a cell source for pharmacology and toxicity testing due to their synthetic and detoxification activities, and because toxicity is a common failure mode in the development of new drugs. However, the use of primary cells is limited by low proliferative capacity, loss of function in vitro, and tendency for dedifferentiation during prolonged culture (ref. 12; incorporated by reference in its entirety). Several strategies to develop hepatocyte-like cells from pluripotent stem cells (iPSC and embryonic stem cells) have been established because of this important role in pharmaceutical testing and discovery (refs. 13-19; incorporated by reference in their entireties). The resulting hepatocyte-like cells express hepatic lineage markers (e.g. cytokeratin-7,8,18, albumin, CYP7A1, hepatocyte nuclear factor-4α) and secrete liver-specific proteins (e.g. albumin, alpha-1-anti-trypsin) yet consistently retain a mixed immature phenotype with expression of alpha fetoprotein (AFP) and decreased overall cellular potency relative to primary hepatocytes, leading to slower metabolism of toxins and small molecules by the cytochrome P450 system (refs. 17, 19-21; incorporated by reference in their entireties). Few synthetic three-dimensional (3D) cell culture systems, such as rudimentary collagen matrices (ref. 22; incorporated by reference in its entirety) or spheroid culture (ref. 23; incorporated by reference in its entirety), recreate cell-cell junctions, and thus liver architecture, more effectively than traditional sandwich culture and lead to enhanced iPSC-hepatocyte function.

### SUMMARY

Provided herein are compositions and systems comprising induced pluripotent stem cell (iPSC)-derived hepatocytes and systems and methods for acellular maturation thereof. In particular, iPSC-derived hepatocytes are matured on decellularized extracellular matrix (ECM) to yield cells with enhanced hepatocytic biomarkers and functionality.

In some embodiments, provided herein are systems, comprising: (a) a bioscaffold comprising acellular hepatic extracellular matrix; and (b) induced pluripotent stem cell (iPSC)-derived hepatocytes. In some embodiments, systems further comprise hepatocyte culture media.

In some embodiments, provided herein are methods of generating mature induced pluripotent stem cell (iPSC)-derived hepatocytes, comprising: (a) providing a bioscaffold derived from a decellularized liver tissue and comprising acellular hepatic extracellular matrix (ECM); (b) seeding the bioscaffold with iPSC-derived hepatocytes; and (c) culturing the iPSC-derived hepatocytes within the bioscaffold under conditions that facilitate maturation of hepatocytes. In some embodiments, conditions that facilitate maturation of hepatocytes comprise substantially atmospheric pressure, about 37° C., and in hepatocyte media.

In some embodiments, provided herein are cells or cell populations comprising hepatocytes produced by the methods and systems herein.

In some embodiments, provided herein are methods of improving liver function in a subject comprising transplanting a cells or cell population comprising hepatocytes produced by the methods and systems herein. In some embodiments, the iPSCs from which the hepatocytes are derived, are in turn derived from somatic cells from the subject. In some embodiments, the iPSCs from which the hepatocytes are derived, are in turn derived from somatic cells from a source other than the subject.

In some embodiments, provided herein are liver assist devices and/or artificial organs comprising a cell or cell population comprising hepatocytes produced by the methods and systems herein. In some embodiments, provided herein is the use of a liver assist device to treat a subject, wherein the iPSCs from which the hepatocytes are derived, are in turn derived from somatic cells from the subject or a source other than the subject.

In some embodiments, provided herein are screening methods comprising exposing a cell or cell population comprising hepatocytes produced by the methods and systems herein to a test agent, and monitoring the effect on the cell population (e.g., compared to a control cell population).

In some embodiments, provided herein is the use of a cell or cell population comprising hepatocytes produced by the methods and systems herein to treat a disease or condition in a subject.

In some embodiments, the cells, systems, devices, and/or methods herein find use in the treatment of, for example: metabolic diseases (e.g., alpha1-antitrypsin deficiency, tyrosinemia, glycogen storage disease (e.g., type 4, type 3, type 2, type 1), maple syrup urine disease (MSUD), Wilson's disease, Neonatal hemochromatosis, Urea cycle deficiency, etc.), Fulminant hepatic failure (e.g., viral, toxin or drug induced, etc.), chronic active hepatitis with cirrhosis, hepatitis B, hepatitis C, autoimmune hepatitis, idiopathic hepatitis, idiopathic neonatal hepatitis, Alagilles syndrome (bile duct paucity syndrome), familial intra-hepatic cholestasis (Byler disease), extra-hepatic biliary atresia, primary sclerosing cholangitis, hepatic tumors (e.g., hepatoblastoma, hepatocellular carcinoma, etc.), cryptogenic cirrhosis, congenital hepatic fibrosis, caroli disease, cystic fibrosis, cirrhosis secondary to prolonged total parenteral nutrition, Budd-Chiari, etc.

### BRIEF DESCRIPTION OF THE DRAWINGS

FIG. 1. Matrix architecture of 3D ECM and bioplotted PLLA-collagen scaffolds. (Panel A) ECM scaffold (left) and porous PLLA-collagen scaffold (right); surface view of the ECM scaffold by (Panel B) Hematoxylin and eosin staining and (Panel C) SEM imaging; (Panels D, E) computer-aided schematic diagram of the PLLA-collagen scaffold showing struts and pores; (Panel F) SEM micrograph of the surface of PLLA-collagen scaffold depicting type 1 collagen infused between PLLA struts (scale bar: 200 μm).

FIG. 2. iPSC-hepatocytes survive and grow within ECM and PLLA-collagen scaffolds. SEM micrographs of recellularized ECM and PLLA-collagen scaffolds with iPSC-hepatocytes over 14 days during an in vitro time course study. Dashed lines delineate boundaries between PLLA bioprinted struts and infused type 1 collagen within 3D bioprinted PLLA-collagen scaffolds. Staining of iPSC-hepatocytes within ECM scaffolds or PLLA-collagen scaffolds shows live cells with minimal dead cells at day 14 (scale bar: 50 μm).

FIG. 3. iPSC-hepatocytes develop cell-cell contacts and biliary canaliculi within ECM scaffolds. Cross sectional view of the ECM scaffold without cells and recellularized ECM scaffolds with iPSC-hepatocytes after 1, 3, 7, and 14 days of in vitro culture by H&E staining (scale bar: 50 μm). iPSC-hepatocytes penetrate into the scaffold over time, indicating migration into the matrix scaffold. inset: higher magnification of iPSC-hepatocytes within ECM scaffolds over time (63×) (scale bar: 20 μm); TEM of iPSC-hepatocytes in the ECM scaffold depicts formation of bile canaliculi-like structures flanked by tight junctions at day 7 (scale bar: BC=biliary canaliculi-like structures, TJ=tight junction, LG=Lipid globule, M=mitochondria).

FIGS. 4A-D. iPSC-hepatocyte culture within 3D scaffolds leads to increased proliferation and expression of mature liver-specific markers. (FIG. 4A) Cell proliferation of iPSC-hepatocytes in ECM scaffolds, PLLA-collagen scaffolds or sandwich controls. Quantitative RT-PCR of metabolic and synthetic genes in iPSC-hepatocytes that either change (FIG. 4B) or remain stable (FIG. 4C) in response to growth in ECM and PLLA-collagen scaffolds compared to growth in standard sandwich control culture at day 14. Fresh, human hepatocytes are shown for comparison. (FIG. 4D) Expression of fetal genes CYP3A7 and AFP in iPSC-hepatocytes in ECM scaffolds or PLLA-collagen scaffolds compared to control sandwich culture at day 14.

FIGS. 5A-C. iPSC-hepatocytes express a higher degree of maturity and albumin synthesis within ECM scaffolds. (FIG. 5A) Albumin production, (FIG. 5B) AFP biosynthesis or (FIG. 5C) albumin/AFP protein synthesis ratio in iPSC-hepatocytes within ECM scaffolds, PLLA-collagen scaffolds or sandwich controls throughout 14 days of culture.

FIGS. 6A-B. ECM scaffolds confer increased P450 activity to iPSC-hepatocytes. (FIG. 6A) CYP2C9, CYP3A4, or CYP1A2 activity in iPSC-hepatocytes within ECM scaffolds, PLLA-collagen scaffolds, or sandwich control environments at 3 days, 7 days, or 14 days of culture; (FIG. 6B) Response of iPSC-hepatocytes to treatment with P450 inducers, rifampicin (CYP2C9 or CYP3A4) or 3-methylcholanthrene (CYP1A2), within ECM scaffolds, PLLA-collagen scaffolds, or sandwich controls at day 14.

FIGS. 7A-B. P450 activity trends higher in iPSC-hepatocytes over time. CYP2C9, CYP3A4 or CYP1A2 activity trends in (FIG. 7A) primary cryo-preserved hepatocytes or (FIG. 7B) iPSC-hepatocytes within ECM scaffolds, PLLA-collagen scaffolds, or sandwich control groups after 3 days, 7 days, and 14 days of culture. CYP activity of primary hepatocytes decreases while CYP activity of iPSC-hepatocytes increases over time, with ECM scaffolds conferring the most robust phenotype for both cell types. (Data is represented as mean±standard deviation. Square: ECM scaffold; triangle: PLLA-collagen scaffold; diamond: sandwich control, * represents a significant difference between ECM scaffolds versus PLLA-collagen scaffolds or sandwich control groups, ** represents a significant difference between ECM scaffolds versus both PLLA-collagen scaffolds and sandwich control groups, † represents a significant difference between PLLA-collagen scaffolds and sandwich control groups).

FIGS. 8A-D. Preparation and characterization of liver ECM. (FIG. 8A) Liver decellularization was carried out by sequential perfusion steps using reagent mixtures depicted in the figure; (FIG. 8B) growth factor content of native and decellularized liver, PLLA-collagen bioscaffold (#), and matrigel-collagen coating (‡) used for the sandwich control (n=4 for each group); (FIG. 8C) Western blot depicting content of ECM proteins within each scaffold environment; (FIG. 8D) H&E staining of native, untreated rat liver and decellularized liver ECM reveals complete lack of cells after decellularization; anti-fibronectin and anti-laminin staining of the native rat liver and the decellularized liver ECM demonstrates that fibronectin and laminin are strongly expressed around vasculature remnants and Glisson's capsule (scale bar: 50 μm) and SEM images of native rat liver and decellularized rat liver ECM depicting a porous ECM scaffold.

FIGS. 9A-C. Baseline characterization of iPSC-hepatocytes: (FIG. 9A) Baseline characterization of fresh iPSC-hepatocytes showing expression of synthetic and metabolic genes that are decreased relative to fresh primary hepatocytes; (FIG. 9B) Albumin synthesis by fresh primary hepatocytes at day 0 compared to iPSC-hepatocytes at day 0; and (FIG. 9C) Decreased baseline activity of P450 isotypes (CYP1A2, CYP2C9 and CYP3A4) in day 0 iPSC-hepatocytes compared to day 0 fresh primary hepatocytes.

FIG. 10. iPSC-hepatocytes develop cell-cell contacts and biliary canaliculi within 3D scaffolds: Low magnification TEM of several adjacent iPSC-hepatocytes in ECM or PLLA-collagen scaffolds at day 7 (left). High magnification of yellow dashed box shows biliary canaliculi-like structures (right) flanked by tight junctions (LG=Lipid globule, BC=biliary canaliculi-like structure, TJ=tight junction, M=mitochondria).

FIGS. 11A-B. Comparison of albumin and AFP expression in iPSC-hepatocytes and primary human hepatocytes: (FIG. 11A) Albumin and (FIG. 11B) AFP biosynthesis in iPSC-hepatocytes relative to cryo-preserved primary hepatocytes after 14 days of culture in sandwich control culture or ECM or PLLA-collagen scaffolds.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “an induced pluripotent stem cell” is a reference to one or more induced pluripotent stem cells, unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein the term “stem cell” (“SC”) refers to cells that can self-renew and differentiate into multiple lineages. A stem cell is a developmentally pluripotent or multipotent cell. A stem cell can divide to produce two daughter stem cells, or one daughter stem cell and one progenitor (“transit”) cell, which then proliferates. Stem cells may be derived, for example, from embryonic sources (“embryonic stem cells”) or derived from adult sources. For example, U.S. Pat. No. 5,843,780 to Thompson describes the production of stem cell lines from human embryos. PCT publications WO 00/52145 and WO 01/00650 describe the use of cells from adult humans in a nuclear transfer procedure to produce stem cell lines.

As used herein, the term “pluripotent cell” or “pluripotent stem cell” refers to a cell that has complete differentiation versatility, e.g., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), a pluriotent cell, even an pluripotent embryonic stem cell, cannot usually form a new blastocyst.

As used herein, the term “induced pluripotent stem cells” (“iPSCs”) refers to a stem cell induced from a somatic cell, e.g., a differentiated somatic cell, and that has a higher potency than said somatic cell. iPSCs are capable of self-renewal and differentiation into mature cells (e.g., endothelial cells).

As used herein the term “feeder cells” refers to cells used as a growth support in some tissue culture systems. Feeder cells may be embryonic striatum cells or stromal cells.

As used herein, the term “hepatocyte” refers to the cells that make up the main parenchymal tissue of the liver (e.g., 70-85% of the liver mass). Hepatocytes are involved in: protein synthesis and storage; transformation of carbohydrates; synthesis of cholesterol, bile salts and phospholipids; detoxification, modification, and excretion of exogenous and endogenous substances; and initiation of formation and secretion of bile

As used herein, the term “iPSC-derived hepatocyte” refers to the hepatocyte cells differentiated from induced pluripotent stem cells, and having the similar gene expression profile, biomarkers (e.g., albumin, alpha-fetoprotein (AFP), alpha-anti-trypsin, annexin I and annexin II, C-reactive protein (CRP), hepatocyte antigen (Hep), hepatocyte paraffin 1 (Hep Par 1), leucine aminopeptidase (LAP), prospero-related homeobox 1 (Prox1), Rex3, tyrosine aminotransferase, tryptophan oxygenase, WT1, etc.) and functions of primary human liver cells.

As used herein, the term “extracellular matrix” (“ECM”) refers to a substance existing in the extracellular space between somatic cells. Extracellular matrices are typically produced by cells, and therefore, are biological materials. Extracellular matrices are involved in supporting tissue as well as in internal environmental structure essential for survival of somatic cells. Extracellular matrices are roughly divided into fibrous components and matrices filling there between. Fibrous components include collagen fibers and elastic fibers. A basic component of matrices is a glycosaminoglycan (acidic mucopolysaccharide), most of which is bound to non-collagenous protein to form a polymer of a proteoglycan (acidic mucopolysaccharide-protein complex). In addition, matrices include glycoproteins, such as laminin of basal membrane, microfibrils around elastic fibers, fibers, fibronectins on cell surfaces, and the like. Examples of components typical to extracellular matrix include, but not limited to, collagen I, collagen III, collagen V, elastin, vitronectin, fibronectin, proteoglycans (for example, decolin, byglican, fibromodulin, lumican, hyaluronic acid, etc.). Various types of extracellular matrix may be utilized in the embodiments herein, although hepatic ECM is particularly preferred.

### DETAILED DESCRIPTION

Provided herein are compositions and systems comprising induced pluripotent stem cell (iPSC)-derived hepatocytes and systems and methods for acellular maturation thereof. In particular, iPSC-derived hepatocytes are matured on decellularized extracellular matrix (ECM) to yield cells with enhanced hepatocytic biomarkers and functionality.

Particularly when compared to defined culture environments, natural liver extracellular matrix (ECM) is biochemically complex, containing more active substrates with a diverse repertoire of proteins with biological function, carbohydrates, and growth factors Experiments conducted during development of embodiments herein demonstrate that ECM mimics the in vivo liver architecture and results in enhanced proliferation, function, and maturation of iPSC-hepatocytes during in vitro culture. Provided herein are structurally complex 3D scaffolds derived from decellularized rat liver ECM (ECM scaffold) using perfusion decellularization (Refs 25-28; incorporated by reference in their entireties). The scaffolds retains the chemical composition of the liver matrix but are small in size (e.g., 5-15 mm) allowing for rapid characterization of evolving cell phenotypes over multiple weeks. To control for the influence of the raw 3D structure on cell-cell interactions versus the ‘outside-in’ signaling capacity of the matrix niche of the ECM scaffold, 3D bioplotting technology was used to print hybrid scaffolds comprised of poly-L-lactic acid (PLLA) coated and infused with type I collagen only (PLLA-collagen scaffold) to represent a ‘deconstructed’ 3D scaffold with structural homogeneity but limited chemical intricacy compared to the ECM scaffold. In experiments described herein, both systems were further compared to the widely used gold-standard control of hepatocyte sandwich culture. Experiments conducted during development of embodiments herein demonstrate that prolonged culture within ECM scaffolds enhances maturation and function of iPSC-hepatocytes compared to growth in PLLA-collagen scaffolds, even though both environments allow for 3D growth and development of cell-cell contacts, indicating that discrete cell-matrix interactions are beneficial for cellular maturation.

iPSCs may be engineered from patient tissue and then differentiated along defined developmental pathways toward maturity. Thus, iPSC-derived cells have the same genetic make-up as the donor patient, making them an ideal cell source for disease modeling, pharmacokinetics and hepatotoxicity testing. Experiments conducted during development of embodiments herein demonstrate that the 3D micro-environment enhances growth, function and maturation of iPSC-hepatocytes without the need for co-culture with a stromal cell population. To evaluate this, two cell-free scaffold systems were compared, the first using natural ECM scaffolds, developed from decellularized rat livers that contain the original pore architecture and overall structure of the whole-organ matrix, and a second, bio-synthetic hybrid PLLA-collagen scaffold developed using 3D bioplotting technology, an advanced rapid-prototyping technique to produce geometrically tunable micron-scale scaffolds. This new technology allowed for the creation of ‘deconstructed’ 3D control environment to investigate mechanism of action by defining the incremental contribution of both 3D architecture and additive matrix function by infusing collagen into the bioplotted scaffold. Moreover, the small scale of both scaffolds provides rapid platform for studies analyzing hepatocyte development and drug efficacy and toxicity.

Experiments demonstrate that decellularized rat ECM is cell-free by histologic and electron microscopy analysis with ˜1% residual DNA remaining compared to the native liver, which is similar to other published decellularization strategies for tissues and organs (refs. 25, 30; incorporated by reference in their entireties). Immunohistochemical and growth factor content analyses of the decellularized ECM further demonstrates preservation of fibronectin and laminin, and ˜50% HGF and FGF. Moreover, ECM scaffolds allowed iPSC-hepatocyte survival, proliferation and migration within this natural matrix.

One limitation of hepatocyte-like cells differentiated from iPSCs and grown using traditional techniques is their decreased phenotypic maturity with lower liver-specific enzyme function compared to primary human hepatocytes (ref 31; incorporated by reference in its entirety). However, iPSC-hepatocytes grown within the ECM scaffolds described herein exhibit increased P450 function. Not only was expression of CYP1A2 (acetaminophen metabolism), CYP2C9 (warfarin metabolism), CYP3A4 (metabolism of ˜50% of medications by the P450 system), and HMGCR transcripts higher in iPSC-hepatocytes grown on ECM scaffolds, but this translated into consistently higher P450 enzyme activity in iPSC-hepatocytes on a day-by-day basis in ECM scaffolds compared to both ‘deconstructed’ 3D controls (e.g. PLLA-collagen) and standard sandwich cultures. Experiments demonstrate that iPSC-hepatocytes display extremely low, and at times undetectable, CYP2C9 activity at initial (day 0) and early time points (˜3 days) in all environments and this continued to be the case throughout the 14-day culture period in standard sandwich cultures. CYP2C9 metabolizes warfarin and phenytoin at varying rates based upon the genetic polymorphism present within each individual. ECM scaffolds led to a 20-fold increase beyond baseline in CYP2C9 activity at day 7. As a model to promote cellular maturation in iPSC-derived cells, 3D environments were tested to enhance proliferation and phenotypic properties of iPSC-derived hepatocytes, a relatively new cell type, and show that growth on ECM scaffolds leads to a population that is closer to primary human hepatocytes with decreased AFP synthesis, increased albumin/AFP ratio, and a trend toward lower expression of fetal-specific markers, AFP and CYP3A7. Together with increasing enzyme function of CYP2C9, CYP3A4 and CYP1A2, this trend provides evidence that growth within ECM scaffolds helps to overcome a primary obstacle of iPSC-hepatocytes by conferring increased maturity and potency to hepatocyte-like cells derived from iPSCs, which may be used to better model disease or analyze metabolism of pharmaceutical compounds.

The use of bioprinting nanotechnology allows 3D comparison environments to evaluate cell phenotype within a multidimensional, but chemically simplified, environment to indicate mechanisms leading to changes in cell function observed in ECM scaffolds. Though clearly distinct from natural liver architecture, 3D bioprinting technology allows development of uniform, tunable scaffolds to evaluate cell growth within defined matrix composition. Experiments demonstrate that bioplotted PLLA-collagen scaffolds provide a 3D environment that permits iPSC-hepatocyte proliferation with increased expression and activity of liver-specific enzymes relative to standard sandwich culture, yet each of these indices were lower in 3D printed scaffolds compared to cells grown in ECM-derived scaffolds. As such, experiments using PLLA-collagen printed scaffolds suggest that growth in 3D and presence of type 1 collagen encourage hepatocyte function, albeit to a limited degree as a reduction in fetal genes CYP3A7 and AFP was not observed in iPSC-hepatocytes grown on PLLA-collagen scaffolds, indicating that both multidimensional growth and appropriate matrix biochemical complexity (as in chemically diverse ECM scaffolds) facilitate maturation of iPSC-hepatocytes.

The use of 3D printed scaffolds allows a degree of comparison to other collagen scaffolding systems such as the Real Architecture for 3D Tissues (ref. 33; incorporated by reference in its entirety) system that uses type 1 collagen as a substrate (ref. 22; incorporated by reference in its entirety). Using this system, it was found albumin secretion to be nearly constant at ˜1.0 μg/day/106 between 10 and 20 days after iPSC differentiation to a common hepatocyte progenitor cell (ref. 22; incorporated by reference in its entirety). In comparison, experiments conducted during development of embodiments herein show that PLLA-collagen scaffolds led to an albumin synthesis rate of ˜3 μg/day/106 and ECM scaffolds led to an albumin synthesis rate of ˜3-4 μg/day/106 between days 7 and 14.

Experiments conducted during development of embodiments herein demonstrate a specific effect of ECM composition on the biochemical function of iPSC-hepatocytes compared to PLLA-collagen scaffolds. ECM scaffolds contain multiple structural proteins including laminin and fibronectin and growth factors that are contributory on a multifactorial level through induction of hepatic nuclear factors and gene networks. Further, in some embodiments, metabolic maturation of iPSC-hepatocytes is provided through co-culture with liver stroma cells, which have been shown to increase catalytic function of iPSC-hepatocytes in biomimetic liver systems (ref. 21; incorporated by reference in its entirety). Experiments conducted during development of embodiments herein also indicate that proliferation, maturation, and hepatic function of iPSC-hepatocytes is further enhanced by producing highly-controlled, fully porous, custom synthetic scaffolds through 3D bioprinting with increased matrix function by ‘adding back’ individual specific ECM components to develop fully synthetic scaffolds that retain the individual, structural building blocks that maximize cell maturation. ECM scaffolds contain FGF, HGF, laminin, fibronectin, and other matrix components that are absent from 3D bioplotted PLLA-collagen scaffolds but may influence maturation of iPSC-hepatocytes. For example, a recent study revealed that a fragment of the ECM protein vitronectin can replace Matrigel to support differentiation of iPSCs into hepatocyte-like cells (ref 34; incorporated by reference in its entirety).

Embodiments herein relate to induced pluripotent stem cells (iPSCs), and the use thereof to derive hepatocytes and ECM-matured hepatocytes. In particular, embodiments herein relate to hepatocytes derived from (differentiated from) iPSCs, and matured on ECM.

In some embodiments, iPSCs are induced using exogenous polypeptides and/or nucleic acids. Induced pluripotent stem cells are cells which have the characteristics of embryonic stem (ES) cells but are obtained by the reprogramming of differentiated somatic cells. Induced pluripotent stem cells have been obtained by various methods. In one method, adult human dermal fibroblasts are transfected with transcription factors Oct4, Sox2, c-Myc and Klf4 using retroviral transduction (Takahashi et al., Cell, 131:861-872, 2007; incorporated by reference in its entirety). The transfected cells are plated on feeder cells (e.g., a mouse cell fibroblast cell line that produces Leukemia Inhibitory Factor (LIF)) in medium supplemented with basic fibroblast growth factor (bFGF). After approximately 25 days, colonies resembling human stem cell colonies appear in culture. The stem cell-like colonies are picked and expanded on feeder cells in the presence of bFGF.

Based on cell characteristics, cells of the stem cell-like colonies are iPSCs. The induced pluripotent stem cells are morphologically similar to human ES cells, and express various human stem cell markers. Also, when grown under conditions that are known to result in differentiation of human stem cells, the iPSCs differentiate accordingly. For example, the induced pluripotent stem cells can differentiate into cells having hepatocyte structures and hepatocyte biomarkers. iPSCs derived from any cell types and by any methods will find use in embodiments herein.

In another method, human fetal or newborn fibroblasts are transfected with four genes, Oct4, Sox2, Nanog and Lin28 using lentivirus transduction (Yu et al., Science, 318:1917-1920, 2007; incorporated by reference in its entirety). At 12-20 days post infection, colonies with stem cell morphology become visible. The colonies are picked and expanded. The induced pluripotent stem cells making up the colonies are morphologically similar to human stem cells (e.g., ES cells), express various human stem cell markers, and form teratomas having neural tissue, cartilage and gut epithelium after injection into mice.

Methods of preparing induced pluripotent stem cells from mouse are also known (Takahashi and Yamanaka, Cell, 126:663-676, 2006; incorporated by reference in its entirety). In some embodiments, induction of iPSC formation requires the expression of or exposure to at least one member from Sox family and at least one member from Oct family. For example, Sox may be Sox-1, Sox-2, Sox-3, Sox-15, or Sox-18; Oct may be Oct-4. Additional factors may increase the reprogramming efficiency, like Nanog, Lin28, Klf4, or c-Myc; specific sets of reprogramming factors may be a set comprising Sox-2, Oct-4, Nanog and, optionally, Lin-28; or comprising Sox-2, Oct4, Klf and, optionally, c-Myc. In certain aspects of the present invention, iPSCs are made from reprogramming somatic cells using reprogramming factors comprising an Oct family member and a Sox family member, such as Oct4 and Sox2 in combination with Klf or Nanog as described above. The somatic cell for reprogramming may be any somatic cell that can be induced to pluripotency, such as a fibroblast, a keratinocyte, a hematopoietic cell, a mesenchymal cell, a liver cell, a stomach cell, or a .beta. cell. In a certain aspect, T cells may also be used as source of somatic cells for reprogramming (see U.S. Application No. 61/184,546, incorporated herein by reference).

The polypeptides (or nucleic acids encoding such polypeptides) used to induce the formation of iPSCs may include any combination of Oct3/4 polypeptides, Sox family polypeptides (e.g., Sox2 polypeptides), KIf family of polypeptides (e.g., Klf4 polypeptides), Myc family polypeptides (e.g., c-Myc), Nanog polypeptides, Lin28 polypeptides, and others understood in the field to be useful for generating iPSCs. For example, in some embodiments, nucleic acid vectors designed to express Oct3/4, Sox2, Klf4, Lin28, and/or c-Myc polypeptides are used to obtain induced pluripotent stem cells. In some cases, polypeptides are directly delivered into target cells to obtain induced pluripotent stem cells using a polypeptide transfection method (e.g., liposome or electroporation). In other embodiments, nucleic acid vectors designed to express iPSC-inuding polypeptides (e.g., Oct3/4, Sox2, Klf4, Lin28, and/or c-Myc polypeptides), are used to obtain induced pluripotent stem cells. Methods and reagents (e.g., polypeptides) for inducing the formation of pluripotent stem cells are not limited to the above, and additional methods and reagents understood in the field are within the scope herein.

In some embodiments, any appropriate cell type is used to obtain induced pluripotent stem cells. In some embodiments, skin, lung, heart, liver, blood, kidney, muscle cells, etc. are used to obtain iPSCs. Such cells can be obtained from any type of mammal including, without limitation, humans, mice, rats, dogs, cats, cows, pigs, or monkeys. In addition, any stage of the mammal can be used, including mammals at the embryo, neonate, newborn, or adult stage.

iPSCs, like ES cells, have characteristic antigens that can be identified or confirmed by immunohistochemistry or flow cytometry, using antibodies for SSEA-1, SSEA-3 and SSEA-4, and TRA-1-60 and TRA-1-81, etc.

In some embodiments, methods are provided for the differentiation/maturation of iPSCs into hepatocytes on scaffolds of extracellular matrix (ECM). In some embodiments, systems comprising iPSCs and/or iPSC-derived hepatocytes on an ECM scaffold are provided. In some embodiments, iPSC-derived hepatocytes, differentiated on ECM matrix, are provided.

The extracellular matrix (ECM) is a collection of extracellular molecules secreted by cells that provides structural and biochemical support to the surrounding cells. Components of the ECM are produced intracellularly by resident cells and secreted into the ECM. Once secreted, they then aggregate with the existing matrix. The ECM is composed of an interlocking mesh of fibrous proteins and glycosaminoglycans (GAGS). Components of the ECM, may include, but are not limited to proteoglycans (e.g., heparan sulfate, chondroitin sulfate, keratan sulfate, etc.), non-proteoglycan polysaccharides (e.g., hyaluronic acid), fibers (e.g., collagens, elastin, etc.), fibronectin, laminin, etc. In some embodiments, hepatic ECM is provided and used as a cellular scaffold. The particular components of hepatic ECM are described, for example, in Martinez-Hernandez &. Amenta. Virchows Arch A Pathol Anat Histopathol. 1993; 423(1):1-11; incorporated by reference in its entirety.

In some embodiments, ECM is derived or obtained from a human or animal subject (e.g., mammal, rodent (e.g., mouse, rat, etc.), bovine, porcine, equine, canine, feline, non-human primate, etc. In some embodiments, ECM is obtained by isolating a section of tissue or organ, and decullularizing the section. In some embodiments, a section of liver tissue is decellularized to obtain hepatic ECM. In some embodiments, tissue is exposed to a decellularization solution or reagent (e.g., 0.1-10% Triton X-100 (e.g., 0.1%, 0.2%, 0.5%, 1%, 2%, 5%, 10%, or ranges therebetween), 0.01-1% NH₄OH (e.g., 0.01%, 0.02%, 0.05%, 0.1%, 0.2%, 0.5%, 1%, or ranges therebetween). In some embodiments, tissue is exposed to Trypsin and/or EDTA/EGTA (Soto-Gutierrez, et al. Tissue Engineering Part C Volume 17, Issue 6 year 2011; incorporated by reference in its entirety).

In some embodiments, a hepatic ECM scaffold, used in embodiments herein, comprises acellular liver extracellular matrix (ECM) and retains the three dimensional architecture and bioactivity of the ECM of the source liver tissue.

Methods for obtaining tissue in general, and liver tissue (e.g., lobe) specifically, are understood within the field. Additionally, methods for decellularization of tissue, while maintaining the components and functionalities of ECM are known within the field. Exemplary methods and reagents that may find use in embodiments herein are described, for example, in Mazza et al. Scientific Reports 5, Article number: 13079 (2015); Crapo et al. Biomaterials. 2011 April; 32(12): 3233-3243; Ye et al. Biomed Mater Eng. 2015; 25(1 Suppl):159-66; Maghsoudlou et al. PLoS ONE 11(5): e0155324; incorporated by reference in their entireties.

In some embodiments, methods and reagents herein are used to differentiate iPSCs into hepatocytes. In some embodiments, iPSCs are differentiated into hepatocytes (e.g., unmatured) or hepatocyte-like cells using existing technologies, such as those described in, for example, WO 2011/116930, U.S. Pat. No. 8,148,151, WO 2004/087896, U.S. Pub. No. 2008/0019949, U.S. Pub. No. 2013/0259836, U.S. Pat. No. 9,057,051, WO 2014/004784, incorporated by reference in their entireties.

In some embodiments, methods and reagents herein are used to mature iPSC-derived hepatocytes (or hepatocyte-like cells) into hepatocytes with enhanced hepatocytic biomarkers and functionality (e.g., more closely mimicking natural hepatocytes than those merely differentiated using traditional techniques). An exemplary protocol is described in detail in Example 1 below. More generally, in some embodiments, ECM scaffolds are placed in a cell culture vessel (e.g., multi-well cell culture plate). In some embodiments, the ECM scaffolds are sterilized (e.g., UV light, 70% ethanol, etc.). In some embodiments, the ECM scaffolds are incubated (e.g., 37° C.) in appropriate culture media for differentiation of iPSCs into hepatocytes (e.g., hepatocyte media (e.g., one or more of: RPMI 1640 medium (Life Technologies, Grand Island, N.Y.) with B27 (Life Technologies, Grand Island, N.Y.), oncostatin M (R&D Systems, Minneapolis, Minn.), dexamethasone (Fisher Scientific, Pittsburgh, Pa.), gentamicin (Life Technologies, Grand Island, N.Y.), etc.). In some embodiments, ECM scaffolds are incubated before seeding in other suitable media. In some embodiments, ECM scaffolds are dried prior to seeding with iPSCs.

In some embodiments, hepatocyte media comprises components selected from, for example, inorganic salts (e.g., calcium nitrate.4H₂O, magnesium sulfate (anhydrous), potassium chloride, sodium bicarbonate, sodium chloride, sodium phosphate dibasic (anhydrous), amino acids (e.g., 1-alanyl-1-glutamine, 1-arginine, 1-asparagine, 1-aspartic acid, 1-cystine.2HCl, 1-glutamic acid, 1-glutamine, glycine, 1-histidine, hydroxy-1-proline, 1-isoleucine, 1-leucine, 1-lysine.HCl, 1-methionine, 1-phenylalanine, 1-proline, 1-serine, 1-threonine, 1-tryptophan, 1-tyrosine.2na.2H₂O, 1-valine, etc.), vitamins (e.g., d-biotin, choline chloride, folic acid, myo-inositol, niacinamide, p-aminobenzoic acid, d-pantothenic acid (hemicalcium), pyridoxine.HCl, riboflavin, thiamine.HCl, vitamin B12, etc.), d-glucose, glutathione, phenol red, etc.

In some embodiments, iPSC-hepatocytes (e.g., hepatocytes or helpatocyte-like cells derived from iPSCs) are placed (e.g., pipetted) onto a scaffold. In some embodiments, between 10³ and 10¹² cells (e.g., 1×10³, 2×10³, 5×10³, 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 2×10¹⁰, 5×10¹⁰, 1×10¹¹, 2×10¹¹, 5×10¹¹, 1×10¹², 2×10¹², 5×10¹², and ranges therebetween (e.g., 1×10⁴-10×10⁸, etc.)) are placed onto/into a scaffold. In some embodiments, cells allowed to attach for a desired time period (e.g., 1-120 minutes (e.g., 1, 2, 5, 10, 20, 30, 40, 50, 60, 90, 120, and ranges therebetween). In some embodiments, the cells and the attached iPSC-hepatocytes are placed into media (e.g., hepatocyte media). In some embodiments, the hepatocytes are cultured in the ECM and hepatocyte media for 1 hour to 20 days (e.g., 1 hour, 2 hours, 4 hours, 6 hours, 8 hours, 12 hours 1 day, 2 days, 4 days, 6 days, 8 days, 10 days, 15 days, 20 days, and ranges therebetween).

In some embodiments, following maturation of the iPSC-hepatocytes in ECM and hepatocyte media, cells exhibit enhanced hepatocytic characteristics (e.g., compared to unmatured iPSC-derived hepatocytes), such as higher hepatocyte biomarker levels (e.g., CYP2C9, CYP3A4, CYP1A2, etc.) and increased liver-specific function. Non-limiting examples of specific enzymes whose levels may be increased through the hepatocyte maturation methods and systems described herein include: ABCB1, ABCB1 1, ABCB4, ABCC1, ABCC2, ABCC3, ABCC4, CYP1A1, CYP1A2, CYP2A6, CYPB6, CYP2C19, CYP2C8, CYP2C9, CYP2D6, CYP3A4, CYP3A43, CYP3A5, CYP3A7, CYP4F12, SLC01A1, SLC2A1, SLCOIBI, SLC02B1, SULT 1A1, SULT1A2, SULT1A3, SULT1B1, SULT1E1, SULT2A1, UGT1A1, UGT1A6, UGT1A8, UGT2B15, UGT2B4, GSTA2, and the like.

In some embodiments, following maturation of the iPSC-hepatocytes in ECM and hepatocyte media, cells exhibit, among other things, enhanced drug metabolism (e.g., evidenced by increased levels of one or more drug metabolism enzymes). Drug metabolism enzymes whose levels can be increased include phase I drug metabolism enzymes, phase II drug metabolism enzymes, and phase III drug metabolism enzymes. Phase I enzymes include cytochrome P450 monooxygenase (CYP) enzymes, flavin-containing enzymes, alcohol dehydrogenase, aldehyde dehydrogenase, monoamine oxidases, NADPH-cytochrome P450 reductases, esterases, amidases, epoxide hydrolases, and the like. Phase II drug metabolism enzymes, which are generally enzymes that catalyze conjugation, include methyltransferases, sulfotransferases (SULT). N-acetyltransferases, bile acid-CoA:amino acid N-acetyltransferases, UDP-glucuronosyltransferases (SULT), glutathione S-transferases (GST), acetyl coA carboxyltransferases, and the like. Phase III drug metabolism enzyme, which generally export compounds from cells, include ATP-binding cassette (ABC) transporters, solute carriers (SLC), and the like.

Hepatocyte function is controlled, in part, by the expression of hepatic transcription factors, e.g., HNF1, HNF4, HNF6, C/EBP alpha, C/EBP beta. In some embodiments, following maturation of the iPSC-hepatocytes in ECM and hepatocyte media, cells exhibit expression of hepatic transcription factors, such as HNF 1, HNF4, HNF6, C/EBP alpha, C/EBP beta, Ahr, CAR, PXR, and RXR.

The iPSC-derived hepatocytes provided by methods and compositions of certain aspects herein find use in a variety of applications. These include but not limited to transplantation or implantation of the hepatocytes in vivo; in vitro screening cytotoxic compounds, carcinogens, mutagens growth/regulatory factors, pharmaceutical compounds, etc.; elucidating the mechanism of liver diseases and infections; studying the mechanism by which drugs and/or growth factors operate; diagnosing and monitoring disease (e.g., cancer) in a patient; gene therapy; the production of biologically active products; etc.

In some embodiments, the iPSC-derived hepatocytes herein find us in the screening of test compounds and other agents. In some embodiments, iPSC-derived hepatocytes differentiated on ECM scaffolds find use in screening for factors (such as solvents, small molecule drugs, peptides, and polynucleotides) or environmental conditions (such as culture conditions or manipulation) that affect the characteristics of hepatocytes.

In some applications, stem cells (differentiated or undifferentiated) are used to screen factors that promote maturation of cells along the hepatocyte lineage, or promote proliferation and maintenance of such cells in long-term culture. For example, candidate hepatocyte maturation factors or growth factors are tested by adding them to stem cells in different wells, and then determining any phenotypic change that results, according to desirable criteria for further culture and use of the cells.

Particular screening applications relate to the testing of pharmaceutical compounds in drug research. In certain aspects, the iPSC-derived hepatocytes herein (e.g., matured on ECM scaffolds) play the role of test cells for standard drug screening and toxicity assays, as have been previously performed on hepatocyte cell lines or primary hepatocytes. Assessment of the activity of candidate pharmaceutical compounds generally involves combining the hepatocytes with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (e.g., by combining with the cells either simultaneously or sequentially), to detect possible drug-drug interaction effects.

In some applications, compounds are screened initially for potential hepatotoxicity (Castell et al., In: In vitro Methods in Pharmaceutical Research, Academic Press, 375-410, 1997; incorporated by reference in its entirety). In some embodiments, cytotoxicity is determined in the first instance by the effect on cell viability, survival, morphology, and leakage of enzymes into the culture medium. In some embodiments, more detailed analysis is conducted to determine whether compounds affect cell function (e.g., gluconeogenesis, ureogenesis, plasma protein synthesis, etc.) without causing toxicity. Lactate dehydrogenase (LDH) is a good marker because the hepatic isoenzyme (type V) is stable in culture conditions, allowing reproducible measurements in culture supernatants after 12-24 h incubation. Leakage of enzymes such as mitochondrial glutamate oxaloacetate transaminase and glutamate pyruvate transaminase can also be used. In some embodiments, a microassay is used for measuring glycogen, which can be used to measure the effect of pharmaceutical compounds on hepatocyte gluconeogenesis (Gomez-Lechon et al., Anal. Biochem., 236:296, 1996; incorporated by reference in its entirety).

Other methods to evaluate hepatotoxicity include determination of the synthesis and secretion of albumin, cholesterol, and lipoproteins; transport of conjugated bile acids and bilirubin; ureagenesis; cytochrome p450 levels and activities; glutathione levels; release of .alpha.-glutathione s-transferase; ATP, ADP, and AMP metabolism; intracellular K⁺ and Ca²⁺ concentrations; the release of nuclear matrix proteins or oligonucleosomes; and induction of apoptosis (e.g., indicated by cell rounding, condensation of chromatin, nuclear fragmentation, etc.). In some embodiments, DNA synthesis i measured as (³H)-thymidine or BrdU incorporation. In some embodiments, effects of a drug on DNA synthesis or structure are determined by measuring DNA synthesis or repair. (³H)-thymidine or BrdU incorporation, especially at unscheduled times in the cell cycle, or above the level required for cell replication, is consistent with a drug effect. Unwanted effects can also include unusual rates of sister chromatid exchange, determined by metaphase spread.

In some embodiments, the iPSC-derived hepatocytes described herein (e.g., matured on ECM scaffolds) find use in liver therapy and/or transplantation. In some embodiments, provided herein is the use of iPSC-derived hepatocytes to restore a degree of liver function to a subject needing such therapy, perhaps due to an acute, chronic, or inherited impairment of liver function.

To determine the suitability of hepatocytes provided herein (e.g., differentiated on ECM scaffolds) for therapeutic applications, the cells can first be tested in a suitable animal model. At one level, cells are assessed for their ability to survive and maintain their phenotype in vivo. Hepatocytes provided herein are administered to immunodeficient animals (e.g., SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether starting cell types such as pluripotent stem cells are still present. In some embodiments, this is performed by providing the administered cells with a detectable label (e.g., green fluorescent protein, or β-galactosidase); or by measuring a constitutive marker specific for the administered cells. Where hepatocytes provided herein are being tested in a rodent model, the presence and phenotype of the administered cells are assessed by immunohistochemistry or ELISA using human-specific antibody, or by RT-PCR analysis using primers and hybridization conditions that cause amplification to be specific for human polynucleotide sequences. Suitable markers for assessing gene expression at the mRNA or protein level are provided in elsewhere in this disclosure. General descriptions for determining the fate of hepatocyte-like cells in animal models is provided in Grompe et al., Sem. Liver Dis., 19:7, 1999; Peeters et al., Hepatology, 25:884, 1997; and Ohashi et al., Nature Med., 6:327, 2000; incorporated by reference in their entireties.

In some embodiments, iPSC-derived hepatocytes (e.g., differentiated on ECM scaffolds) provided herein are assessed for their ability to restore liver function in an animal lacking full liver function See, e.g., Braun et al., Nature Med., 6:320, 2000; Rhim et al., Proc. Natl. Acad. Sci. USA, 92:4942, 1995; Lieber et al., Proc. Natl. Acad. Sci. USA, 92:6210, 1995; Mignon et al., Nature Med., 4:1185, 1998; Overturf et al., Human Gene Ther., 9:295, 1998; Rudolph et al., Science, 287:1253, 2000; incorporated by reference in their entireties).

In some embodiments, hepatocytes that demonstrate desirable functional characteristics according to their profile of metabolic enzymes, or efficacy in animal models, are suitable for direct administration to human subjects (e.g., subjects with impaired liver function). In some embodiments, for purposes of hemostasis, the cells are administered at any site that has adequate access to the circulation, typically within the abdominal cavity. For some metabolic and detoxification functions, it is advantageous for the cells to have access to the biliary tract. Accordingly, the cells are administered near the liver (e.g., in the treatment of chronic liver disease) or the spleen (e.g., in the treatment of fulminant hepatic failure). In some methods, the cells are administered into the hepatic circulation either through the hepatic artery, or through the portal vein, by infusion through an in-dwelling catheter. In some embodiments, a catheter in the portal vein is manipulated so that the cells flow principally into the spleen, or the liver, or a combination of both. In another method, the cells are administered by placing a bolus in a cavity near the target organ, typically in an excipient or matrix that will keep the bolus in place. In another method, the cells are injected directly into a lobe of the liver or the spleen.

In certain embodiments, iPSC-derived hepatocytes provided herein are used for therapy of a subject in need of having hepatic function restored or supplemented. Human conditions that may be appropriate for such therapy include fulminant hepatic failure due to any cause, viral hepatitis, drug-induced liver injury, cirrhosis, inherited hepatic insufficiency (such as Wilson's disease, Gilbert's syndrome, or α₁-antitrypsin deficiency), hepatobiliary carcinoma, autoimmune liver disease (such as autoimmune chronic hepatitis or primary biliary cirrhosis), and any other condition that results in impaired hepatic function. For human therapy, the dose is generally between about 10⁶ and 10¹⁵ cells (e.g., 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 2×10¹⁰, 5×10¹⁰, 1×10¹¹, 2×10¹¹, 5×10¹¹, 1×10¹², 2×10¹², 5×10¹², 1×10¹³, 2×10¹³, 5×10¹³, 1×10¹⁴, 2×10¹⁴, 5×10¹⁴, 1×10¹⁵, 2×10¹⁵, 5×10¹⁵, or ranges therebetween (e.g., 10⁹-10¹²), making adjustments for the body weight of the subject, nature and severity of the affliction, and the replicative capacity of the administered cells. The ultimate dose determination with the clinician.

In some embodiments, the iPSC-derived hepatocytes (e.g., differentiated on ECM scaffold) find use in a liver assist device. Certain aspects of this invention include hepatocytes provided herein that are encapsulated or part of a bioartificial liver device. Hepatocytes provided in certain aspects of this invention are encapsulated according to such methods for use either in vitro or in vivo.

Bioartificial organs for clinical use are designed to support an individual with impaired liver function—either as a part of long-term therapy, or to bridge the time between a fulminant hepatic failure and hepatic reconstitution or liver transplant. Bioartificial liver devices are described and exemplified in U.S. Pat. Nos. 5,290,684, 5,624,840, 5,837,234, 5,853,717, 5,935,849, incorporated by reference in their entireties. Suspension-type bioartificial livers comprise cells suspended in plate dialysers, microencapsulated in a suitable substrate, or attached to microcarrier beads. In some embodiments, iPSC-derived hepatocytes (e.g., differentiated on ECM scaffolds) are placed on a solid support in a packed bed, in a multiplate flat bed, on a microchannel screen, or surrounding hollow fiber capillaries. In some embodiments, the device has an inlet and outlet through which the subject's blood is passed. In some embodiments, the device has a set of ports for supplying nutrients to the cells. In some embodiments, the hepatocytes are maintained on ECM (e.g., the ECM scaffolds described herein).

In some embodiments, liver assist devices comprising iPSC-derived hepatocytes (e.g., differentiated on ECM scaffolds) are used to detoxify a fluid such as blood, wherein the fluid comes into contact with the hepatocytes under conditions that permit the cell to remove or modify a toxin in the fluid. In some embodiments, the detoxification involves removing or altering at least one ligand, metabolite, or other compound (either natural and synthetic) that is usually processed by the liver. Such compounds include but are not limited to bilirubin, bile acids, urea, heme, lipoprotein, carbohydrates, transferrin, hemopexin, asialoglycoproteins, hormones like insulin and glucagon, and a variety of small molecule drugs. In some embodiments, devices are used to enrich the efferent fluid with synthesized proteins such as albumin, acute phase reactants, and unloaded carrier proteins. In some embodiments, devices are optimized so that a variety of functions is performed, thereby restoring as many hepatic functions as are needed. In some embodiments, in the context of therapeutic care, a device processes blood flowing from a patient in hepatocyte failure, and then the blood is returned to the patient.

For purposes of manufacture, distribution, and use, the iPSC-derived hepatocytes described herein (e.g., differentiated on ECM scaffolds) are typically supplied in the form of a cell culture or suspension. In some embodiments, the cells are maintained on the scaffolds described herein. In some embodiments, the cells are removed from the ECM scaffolds used for differentiation and are maintained in another vessel of on a different scaffold. In some embodiments, cells are maintained in an isotonic excipient or culture medium, optionally frozen to facilitate transportation or storage.

In some embodiments, different reagent systems are provided, comprising a set or combination of cells that exist at any time during manufacture, distribution, or use. The cell sets comprise any combination of two or more cell populations described in this disclosure, exemplified but not limited to iPSCs, iPSC-derived hepatocytes, precursors thereof, subtypes thereof, etc. in combination with undifferentiated stem cells, somatic cell-derived hepatocytes, or other differentiated cell types. In some embodiments, cell populations in the set(s) share the same genome or a genetically modified form thereof. Each cell type in the set may be packaged together, or in separate containers in the same facility, or at different locations, at the same or different times, under control of the same entity or different entities sharing a business relationship.

### EXPERIMENTAL

### Example 1

### Materials and Methods

### Development of ECM and Bioplotted Scaffolds

ECM scaffolds: Male Sprague-Dawley rats weighing 220-250 g (Charles River Laboratories, Wilmington, Mass.) were cared for in accordance with protocols approved by the Institutional Animal Care and Use Committee of Northwestern University. After adequate induction of anesthesia, 2×10³ unit/kg body weight of heparin was injected intravenously. The liver was isolated and perfused with 20 ml of cold saline and decellularized by perfusion of agents through a portal vein cannula: deionized water, followed by the decellularization solution (1% Triton X-100 and 0.1% NH₄OH), and rinsed with deionized water (ref. 26; incorporated by reference in its entirety). To prepare ECM scaffolds, decellularized liver lobes were embedded within optical cutting temperature (OCT) compound, flash frozen, sectioned to a thickness of 500 μm, and biopsy punched into 8 mm-diameter disks (Miltex, York, Pa.).

PLLA-collagen scaffolds: A 3D-Bioplotter (EnvisionTEC GmbH, Germany) was used to fabricate PLLA-collagen scaffolds (8 mm-diameter, 500 um-thick). PLLA (PURAC Biomaterials, Denmark) was melted at 220° C., 3D-printed via hot-melt extrusion onto a stage heated to 60° C., subsequently treated with 70% ethanol to reduce inherent hydrophobicity and induce surface hydrophilicity, and infused with 0.05 wt. % type I bovine collagen solution in 50 mM acetic acid. Infused scaffolds were immediately frozen at −80° C. for several hours prior to lyophilization.

### Cells and Culture Conditions

Individual scaffolds were placed in a 48-well cell culture plate (Life Technologies, Grand Island, N.Y.), sterilized with 70% ethanol, and incubated at 37° C. overnight in hepatocyte medium (RPMI 1640 medium (Life Technologies, Grand Island, N.Y.) with B27 (Life Technologies, Grand Island, N.Y.), 20 ng/ml oncostatin M (R&D Systems, Minneapolis, Minn.), 0.1 μM dexamethasone (Fisher Scientific, Pittsburgh, Pa.), and 25 μg/ml gentamicin (Life Technologies, Grand Island, N.Y.)). Scaffolds were dried for five minutes prior to cell seeding.

Human iPSC-hepatocytes (iCell Hepatocytes) were received fresh, in suspension within a T-flask at room temperature from Cellular Dynamics International (Madison, Wis.). All cells were differentiated from a single donor iPSC clone and results from multiple production (differentiation) runs were found to yield consistent results. Cells were grown and handled according to the manufacturer's recommendations. A 20 μl cell suspension containing 1×10⁶ cells was pipetted onto each scaffold and cells were allowed to attach for 20 minutes. Next, 10 of each scaffold type with cells were transferred into a T25 flask, 10 ml of hepatocyte medium (In Vitro Technologies Inc., Baltimore, Md.) was added, and scaffolds were maintained under dynamic culture conditions at 37° C. and 5% CO₂ on an orbital shaker (Stovall Life Science, Greensboro, N.C.) set at 1 rotation/sec to prevent settling of scaffolds on the bottom of the flask while minimizing foam formation on the surface of the media.

Sandwich control group: 0.5×10⁶ iPSC-hepatocytes were seeded onto 24-well polystyrene plates coated with a type 1 collagen gel (BD Biosciences, San Jose, Calif.). 0.5 ml hepatocyte medium was added to maintain the same cell:media ratio used in micro-scaffold culture conditions. The following day, 0.25 mg/ml growth factor reduced Matrigel® Matrix (BD Biosciences, San Jose, Calif.) was placed over the cells.

Fresh primary human hepatocytes expanded in triple knockout Fah^{-/-}/Rag2^{-/-}/Il2rg^{-/-} (FRG) mice (Yecuris Inc., Tualatin, Oreg.) or cryo-preserved primary human hepatocytes (male, age 56, In Vitro Technologies Inc., Baltimore, Md.) were adopted as positive controls. Primary hepatocytes in hepatocyte medium were seeded onto the ECM, PLLA-collagen scaffolds, or type 1 collagen coated polystyrene plates with growth factor reduced Matrigel® overlay following the same method described for iPSC-hepatocytes. Cell culture medium was changed every other day for all groups.

### DNA Quantification and Quantitative RT-PCR

The amount of DNA remaining after cell removal was used to evaluate the degree of decellularization in acellular scaffolds compared to the normal (untreated) organ. The tissue was completely dehydrated and DNA was extracted and subsequently purified using a standard kit (Qiagen, Gaithersburg, Md.). DNA concentration was measured on a nanodrop machine at 260 nm, and represented as ng DNA/mg dry-weight of the sample.

To quantify cell proliferation within scaffolds after 1, 3, 7, and 14 days in culture, cell-laden scaffolds were digested using 0.1% Triton X-100 for 30 minutes followed by 1 minute ultrasonic treatment (50 Hz, Branson Ultrasonics Corporation, Danbury, Conn.). DNA within resulting lysates was measured by reading fluorescein at 485/535 nm using Quant-iT™ PicoGreen kit (Life Technologies, Grand Island, N.Y.). Known concentrations of iPSC-hepatocytes and primary hepatocytes were used to construct a standard curve and correlate DNA concentration to cell number.

A two-step quantitative real-time reverse transcription polymerase chain reaction (qRT-PCR) was performed at day 14. Total RNA was isolated using the TRI Reagent® solution (Life Technologies, Grand Island, N.Y.). Reverse transcription using the High Capacity RNA-to-cDNA Kit (Life Technologies, Grand Island, N.Y.) was performed. The mRNA expression of each target gene was normalized to cyclophilin expression. Primer sequences are shown in Table 1 qPCR was carried out with iQ™ SYBR® Green and detected using the iQ™5 Optical System (Bio-Rad, Des Plaines, Ill.). The thermal profile used was 50° C. for 2 min, 95° C. for 10 min, 40 amplification cycles of 95° C. for 60 s, and 58° C. for 60 s. The relative degree of gene amplification was calculated using 2^{̂(Ct gene 2-Ct Cyclophilin2)-(Ct gene 1-Ct Cyclophilin 1)}. “Ct gene 1” represents the threshold cycle (Ct) of the target gene of the reference population and “Ct gene 2” represents the target gene in the sample of interest.

TABLE 1
Primers used in Quantitative RT-PCR:

| | |
|---|---|
| Cyclophilin | F(forward)-CACAGGAGGAAAGAGCATCTAC |
| | (SEQ ID NO: 1) |
| | R(reverse)-CACAGACGGTCACTCAAAGAA |
| | (SEQ ID NO: 2) |
| HMGCR | F-CCTGTTGGAGTGGCAGGACCC |
| | (SEQ ID NO: 3) |
| | R-TGGTGCTGGCCACAAGACAACC |
| | (SEQ ID NO: 4) |
| ALB | F-AGCATGGGCAGTAGCTCGCCT |
| | (SEQ ID NO: 5) |
| | R-AGGTCCGCCCTGTCATCAGCA |
| | (SEQ ID NO: 6) |
| CYP2C9 | F-GGATTTGCCTCTGTGCCGCC |
| | (SEQ ID NO: 7) |
| | R-GCAGCCAGGCCATCTGCTCTT |
| | (SEQ ID NO: 8) |
| CYP1A2 | F-TCCCAGTCTGTTCCCTTCTCGGC |
| | (SEQ ID NO: 9) |
| | R-TTGACAGTGCCAGGTGCGGG |
| | (SEQ ID NO: 10) |
| GSTA1 | F-GTTTCTACAGCCTGGCAGCCCA |
| | (SEQ ID NO: 11) |
| | R-AGTTCTTGGCCTCCATGACTGC |
| | (SEQ ID NO: 12) |
| NDUFA3 | F-CGTGTCCTTCGTCGTCGGGG |
| | (SEQ ID NO: 13) |
| | R-CTGGGCACGTCGGGCATGTT |
| | (SEQ ID NO: 14) |
| AFP | F-GCTGACCTGGCTACCATATTT |
| | (SEQ ID NO: 15) |
| | R-GGGATGCCTTCTTGCTATCTC |
| | (SEQ ID NO: 16) |
| CYP3A7 | F-CTGAGAAGTTCCTCCCTGAAAG |
| | (SEQ ID NO: 17) |
| | R-GCACGTACAGAATCCCTGATTA |
| | (SEQ ID NO: 18) |
| CYP3A4 | F-CTGCTTCTCACGGGACTATTT |
| | (SEQ ID NO: 19) |
| | R-CCTCCCAAACTGCTAGGATTAC |
| | (SEQ ID NO: 20) |

### Immunohistochemical Characterization, Scanning Electron Microscopy, and Transmission Electron Microscopy

ECM specimens were fixed in 4% paraformaldehyde and stained with hematoxylin and eosin (H&E). For immunohistochemistry staining, after rehydration and antigen retrieval, sections were incubated at 4° C. overnight with anti-laminin B2 gamma 1 antibody (1:100, Abcam, Cambridge, Mass.) or anti-fibronectin antibody (1:100, Santa Cruz Biotechnology, Santa Cruz, Calif.). The secondary antibody (goat anti-mouse IgG HRP, 1:500, Abcam, Cambridge, Mass.) was incubated at room temperature for 60 minutes. Visualization of the immunohistochemical reaction was performed with DAB/H₂O₂ solution (Nichirei, Japan) and counter-stained with Mayer's hematoxylin. H&E and immunohistochemical characterization could not be performed on PLLA-collagen specimens due to sensitivity to histological solvents and resulting inability of the sample to remain fixed to glass slides during the dehydration processes.

To assess cell viability, both ECM and PLLA-collagen scaffolds were subjected to live-dead staining using a live/dead kit (Life Technologies, Grand Island, N.Y.) and observed with the Nikon C2 confocal laser scanning system. For SEM, samples were fixed in a 5 wt % glutaraldehyde for 4 hours, dehydrated in a graded ethanol series, critically point dried, coated with osmium, and observed by SEM (LEO Gemeni 1525). For TEM, specimens were fixed with 2.5% glutaraldehyde, subsequently post-fixed with 1% osmium tetroxide (1 hour), with 1% uranyl acetate in maleate buffer (1 hour), dehydrated with graduated concentrations of ethanol and propylene oxide, and then embedded in Epon (polymerized at 60° C. for 48 h). Ultra-thin specimens were observed using Hitachi HT-7700 Biological TEM.

### Measurement of CYP1A1, CYP2C9, and CYP3A4 Enzyme Activities

Cells seeded in sandwich control culture, ECM, and PLLA-collagen scaffolds treated with or without induction agents (5 μM 3-methylcholanthrene for CYP1A2 or 50 μM rifampicin for CYP2C9 and CYP3A4) for 48 h were incubated with 100 μM Luc-ME (CYP1A2) for 0.5 h, 100 μM Luc-H (CYP2C9) for 4 h, or 3 μM Luc-IPA (CYP3A4) (Promega, Madison Wis.) for 0.5 h. 100 μl of this luciferin medium was added to a 96-well white opaque plate with 100 μl luciferin detection reagent, and the luminescence signal was measured on a Cytation 3 Cell Imaging Multi-Mode Reader (BioTek Inc. Winooski, Vt.). After determining the activity, the amount of cells in each scaffold or sandwich culture was analyzed with Quant-iT™ Pico Green kit following the protocols described above.

### Statistical Analysis

Experimental data are presented as mean±standard deviation. A one-way analysis of variance (ANOVA) was used for statistical analysis, with the Holm-Sidak test used for post hoc pair-wise comparisons and testing against the control group. The Student's t-test was applied for two-group comparisons using SPSS and Microsoft Excel software. Differences were considered statistically significant when p≦0.05.

### Western Blot Analysis; Growth Factor, Albumin, and AFP Detection

Growth factor content was determined in both the native and decellularized ECM scaffolds, as well as the 0.25 mg/ml growth factor reduced MATRIGEL overlay and the type 1 collagen used to make sandwich and PLLA-collagen environments (ref. 37; incorporated by reference in its entirety). The concentration of basic fibroblast growth factor (bFGF) and hepatocyte growth factor (HGF) in urea-heparin extracts was evaluated with the respective enzyme-linked immunosorbent assay (ELISA) Kit (R&D Systems, Minneapolis, Minn.) per the manufacturer's protocols.

The concentration of human albumin and alpha fetoprotein (AFP) secreted into the culture medium was determined in the culture media collected from each flask containing scaffolds or from each well of the sandwich control group before medium exchange. Samples were centrifuged at 500 g to separate debris. Albumin and AFP were measured by an albumin ELISA kit (Bethyl Laboratories, Montgomery Tex.) or an AFP human ELISA kit (Abcam, Cambridge, Mass.), respectively. Albumin and AFP synthesis is expressed as the amount of albumin or AFP produced per 10⁶ cells per day using the total cell number per bioscaffold at each time point obtained from the Picogreen measurement of cell density.

The presence of ECM-associated proteins was determined by western blot analysis of individually homogenized scaffolds or Matrigel and type 1 collagen in lysis buffer (Thermo Scientific, Pittsburgh, Pa.) containing 2% Halt phosphatase inhibitor (Thermo Scientific, Pittsburgh, Pa.) and 5% protease cocktail inhibitor (Sigma Aldrich, St. Louis, Mo.). Protein concentrations were quantitated and normalized using a bicinchoninic acid assay (Sigma Aldrich, St. Louis, Mo.). Primary antibodies for laminin B2 gamma 1, fibronectin, or type 1 collagen (all at 1:2000, Abcam, Cambridge, Mass.) were used. Bound antibody was revealed with goat anti-mouse IgG (HRP) or goat anti-rabbit IgG (HRP) (1:10000, Abcam, Cambridge, Mass.) and developed using Amersham ECL Prime Western Blotting Detection Reagent (GE Healthcare Biosciences, Pittsburgh, Pa.).

### Example 2

### Results

### Development and Characterization of 3D Liver ECM Bioscaffolds

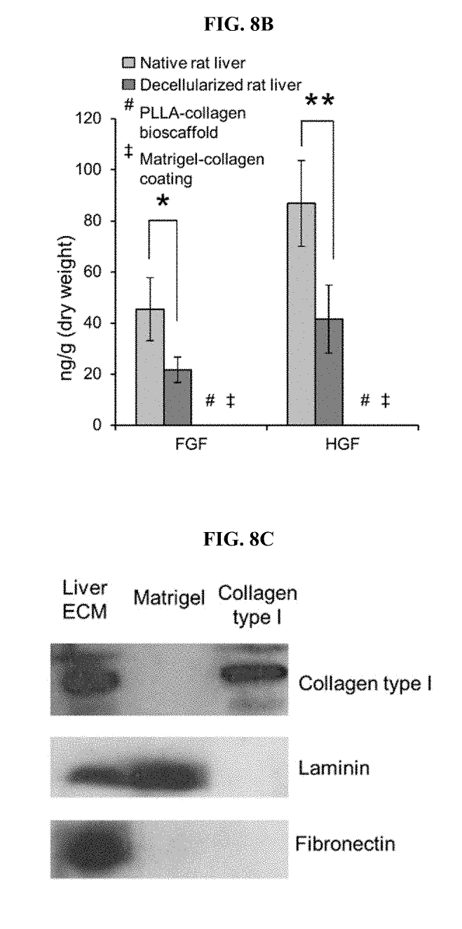

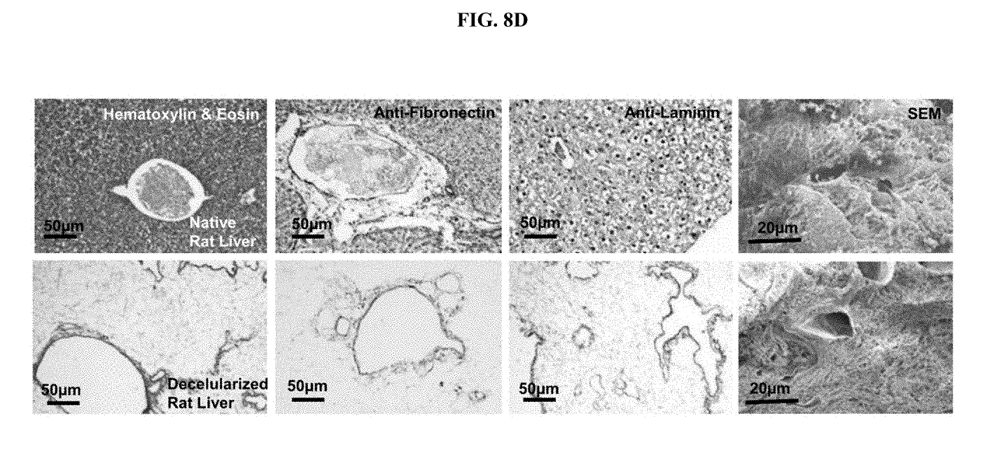

Acellular ECM scaffolds were developed by sequential perfusion of weak detergents through the liver vasculature (FIG. 8A). The resulting scaffold was opaque in appearance (FIG. 8A), and quantitative DNA analysis revealed a 98.9% reduction in DNA content following decellularization (native liver: 6163.7±1221.6 ng/mg; decellularized ECM: 67.9±7.7 ng/mg, p<0.01,). Despite the near-absence of DNA, indicating removal of the cellular compartment, growth factors remained immobilized to structural proteins of the ECM: the content of hepatocyte growth factor (HGF) was 41.61±13.36 ng/g in the decellularized liver matrix and 86.89±16.76 ng/g in native, untreated liver; the content of basic fibroblast growth factor (bFGF) was 21.80±5.02 ng/g in the decellularized liver scaffold and 45.50±12.36 ng/g in the native, untreated liver (FIG. 8B). These results indicate that ˜50% of HGF and bFGF were preserved after decellularization, similar to scaffolds developed using other cell-removal strategies (ref 25; incorporated by reference in its entirety). Fibronectin, laminin, and type I collagen proteins were further detected in the liver ECM by western blot (FIG. 8). Scanning electron microscopy (SEM) and hematoxylin & eosin staining of the decellularized liver matrix also revealed the acellularity of liver ECM with preservation of the 3D lacunae structure (FIG. 8D). Immunohistochemical characterization of the liver ECM (FIG. 8D) further confirmed matrix content and found laminin and fibronectin to be more prevalent around vessel remnants and Glisson's capsule.

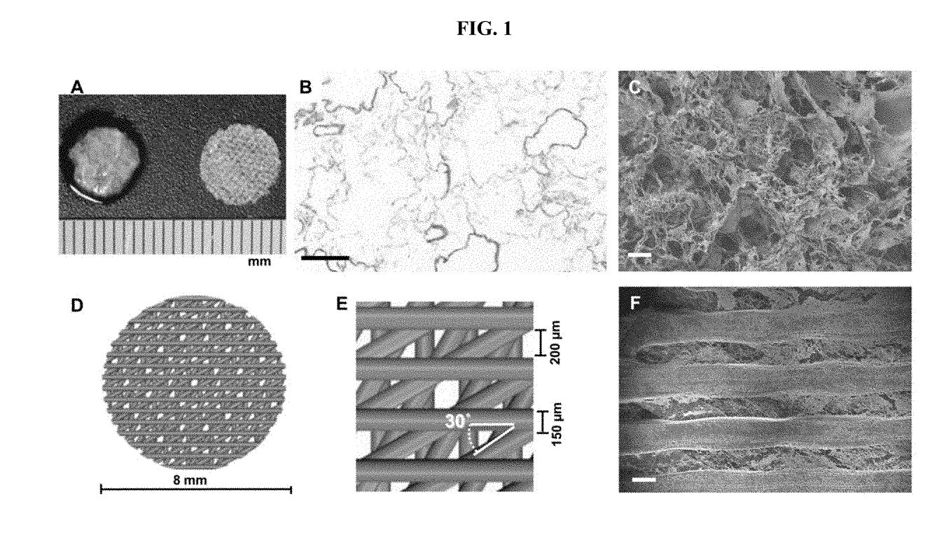

Individual ECM scaffolds (FIG. 1A left), measuring 8 mm in diameter, were developed from the decellularized liver matrix, and preservation of the ECM porous micro-structure was revealed by H&E staining (FIG. 1B) and SEM imaging (FIG. 1C). As a comparison matrix, a bio-hybrid PLLA-collagen scaffold was also developed (FIG. 1A right) as a ‘deconstructed’ 3D environment possessing an open pore structure and containing only type 1 collagen without growth factors (FIG. 8B) or other structural proteins (FIG. 8C). These scaffolds are comprised of four 125 μm-thick layers (FIG. 1D). Each layer is comprised of parallel PLLA struts measuring 150 μm wide and spaced 200 μm apart with each successive layer oriented 30° with respect to the previous layer to mimic the porous nature of the liver ECM albeit with a more homogenous architecture and thicker strut size afforded by the 3D bioprinting process (FIG. 1E). Infused collagen fibers are observed between PLLA struts by SEM (FIG. 1F).

### Cell Viability, Proliferation, and Migration

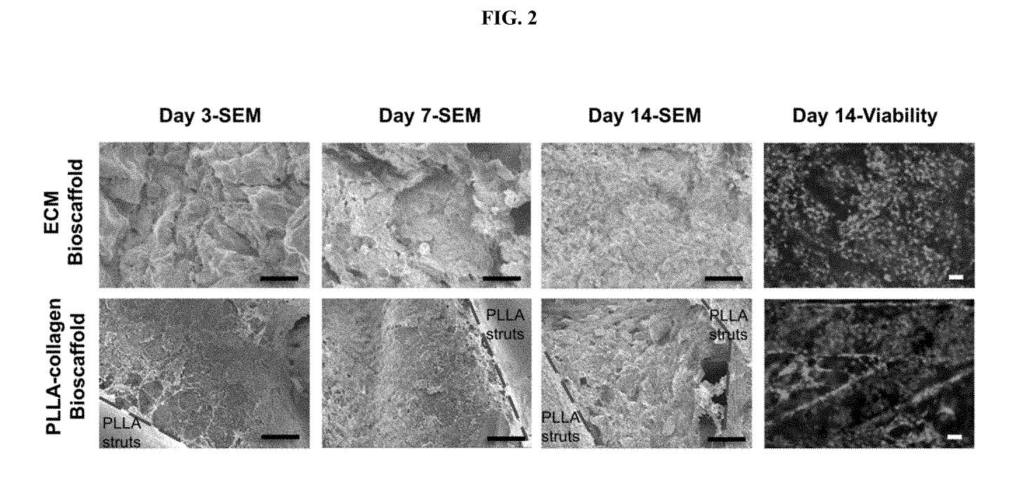

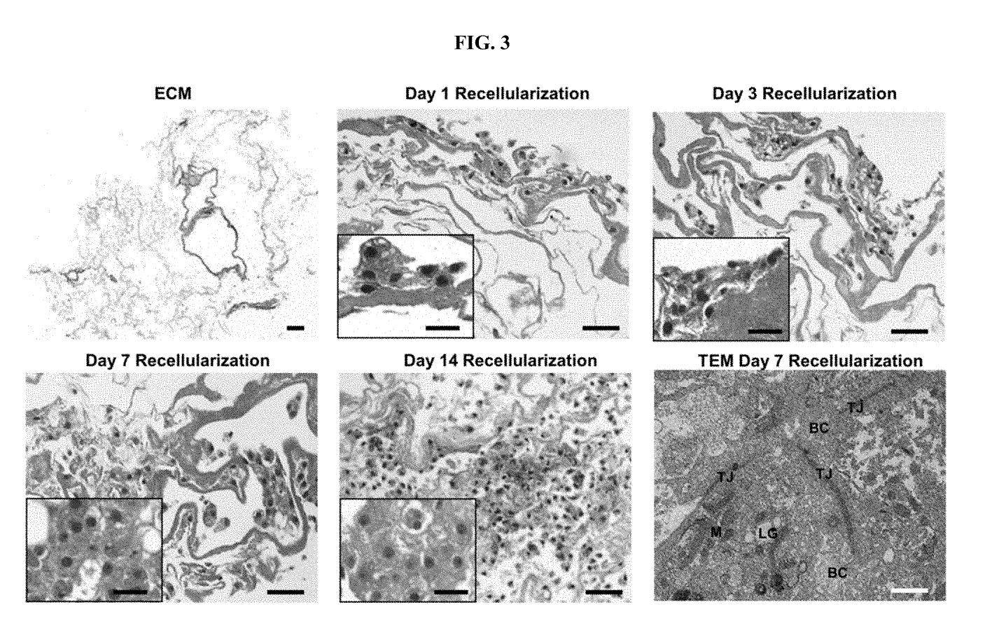

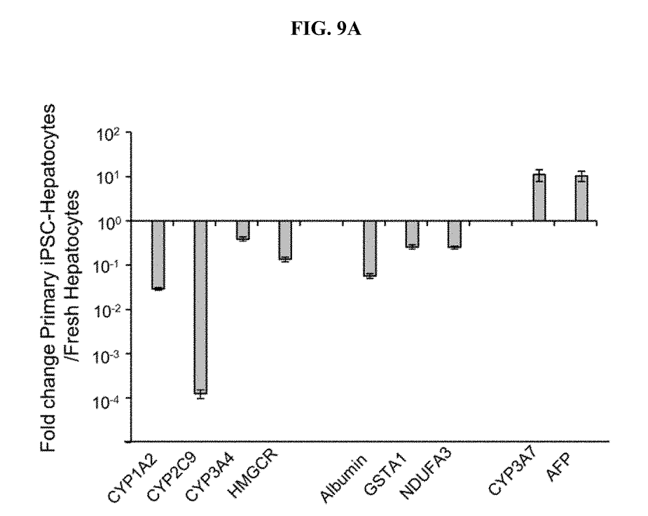

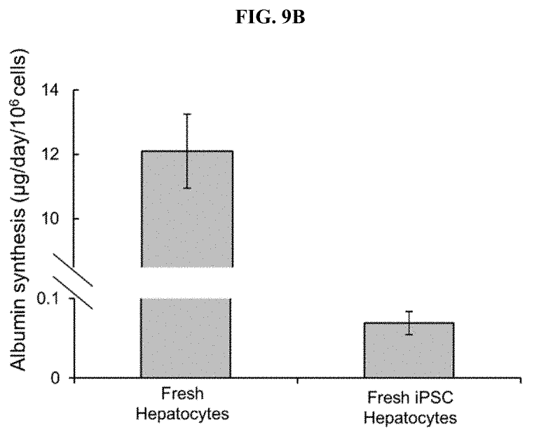

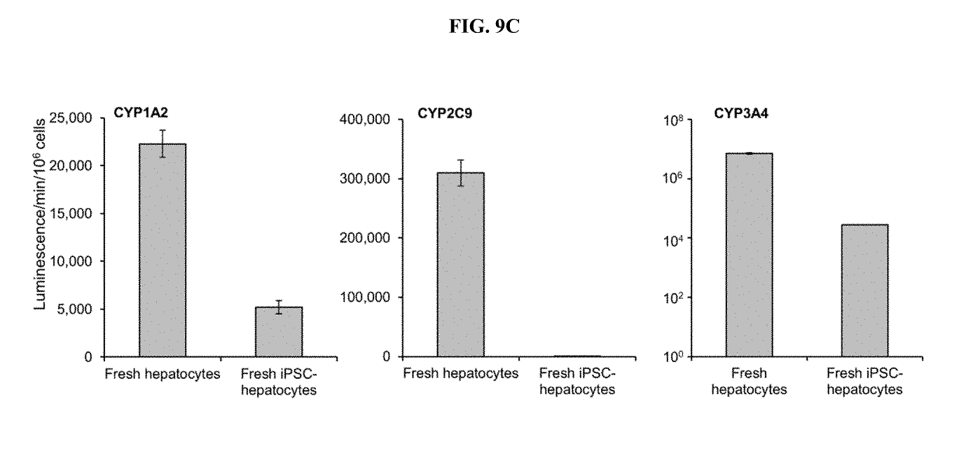

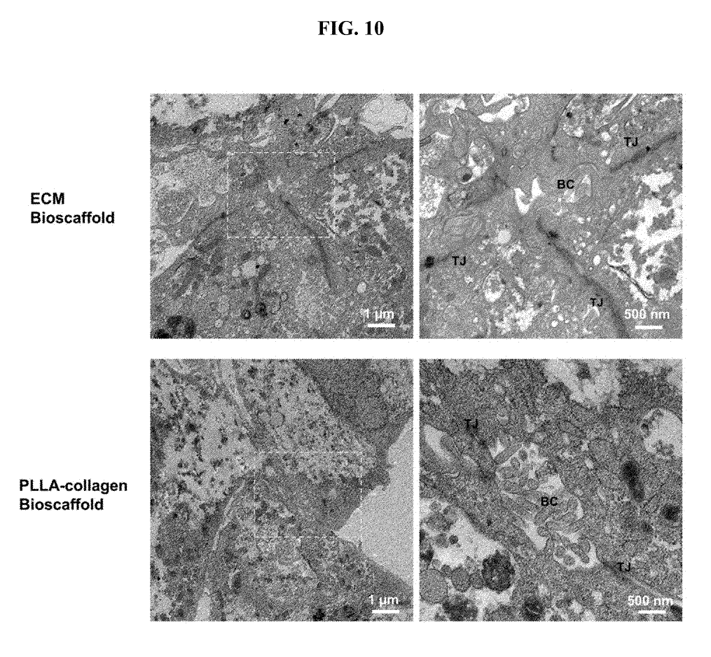

Phenotyping of commercially available, differentiated iPSC-hepatocytes was performed to establish a baseline and assess initial hepatocyte-like characteristics compared to fresh primary hepatocytes. It was found hepatocyte-like cells that were differentiated from iPSCs express genes signifying hepatocyte lineage differentiation, albeit at a lower level (FIG. 9A) with poorer synthetic function (albumin production, FIG. 9B) and lower P450 enzyme activity (FIG. 9C) compared to fresh primary human hepatocytes. Fetal hepatocyte markers, AFP and CYP3A7, were increased in iPSC-hepatocytes compared to fresh primary hepatocytes. To assess the ability of the cell-free scaffolds described herein to influence iPSC-hepatocyte function and maturation, cells were seeded onto ECM or PLLA-collagen scaffolds or grown between a type 1 collagen monolayer and Matrigel overlay in standardized control sandwich culture. SEM imaging of cell-laden scaffolds shows abundant iPSC-hepatocytes within the interconnecting pore structure of the collagen network in PLLA-collagen scaffolds and within ECM scaffolds for two weeks (FIG. 2). Live/dead staining of iPSC-hepatocytes within scaffolds reveals that cells remained viable and evenly distributed across scaffolds after 14 days of culture (FIG. 2). H&E staining of ECM scaffolds in cross-section depicts cell attachment at day 1 and multi-layered cell attachment after day 3 with penetration and migration into the matrix and formation of cell-cell connections on day 7, which continued through day 14 (FIG. 3). Bile canaliculi-like structures and formation of tight junctions between adjacent iPSC-hepatocytes were observed on both ECM (FIGS. 3 and 10) and PLLA-collagen (FIG. 10) scaffolds by 7 days.

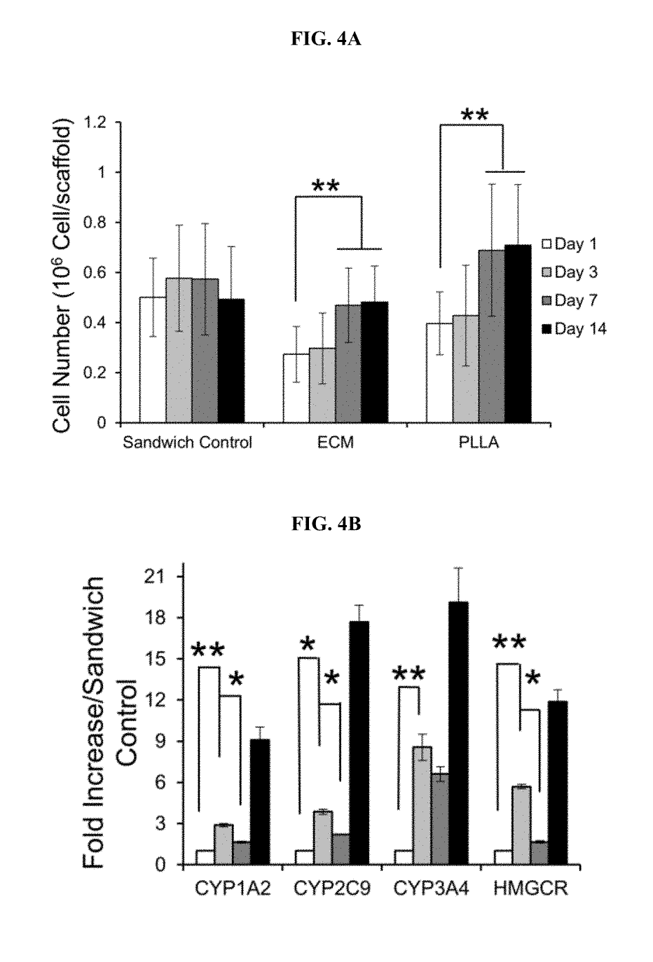

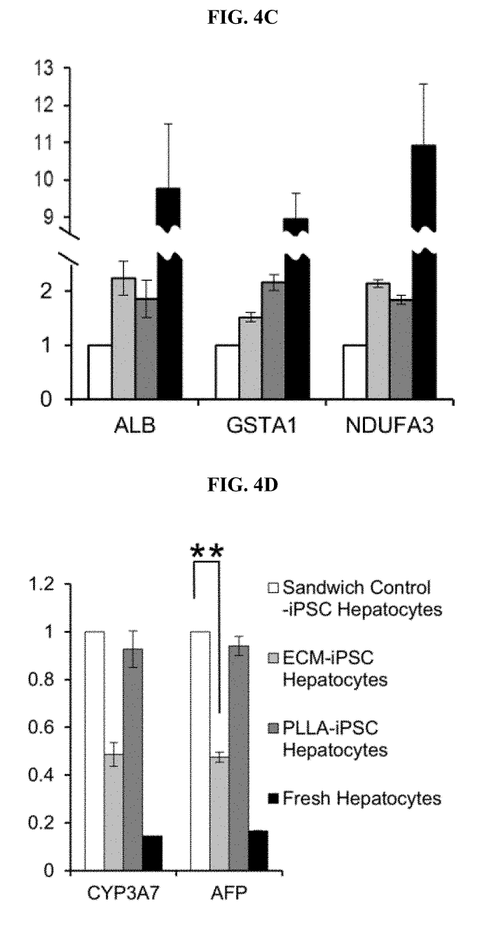

Total DNA content was used to determine cell density and proliferation of iPSC-hepatocytes in ECM, PLLA-collagen scaffold, and sandwich control groups at day 1, and was found to be 0.27±0.11×10⁶, 0.39±0.13×10⁶, and 0.50±0.15×10⁶ cells/scaffold, respectively. Cell density in both engineered scaffolds increased during the first 7 days, with a 1.7-fold increase in iPSC-hepatocytes grown in the ECM scaffold and a 1.7-fold increase in the PLLA-collagen scaffold. The cell density of the sandwich control group did not show any significant change indicating absence of proliferation during the 14-day culture period (FIG. 4A).

### Increased Expression of Hepatocyte-Specific Markers in ECM Scaffolds

mRNA transcripts encoding phase I and II xenobiotic enzymes (CYP1A2, CYP2C9, CYP3A4, and HMGCR) assessed on day 14 were higher in iPSC-hepatocytes grown in either ECM or PLLA-collagen scaffolds compared to the sandwich control group. Expression of CYP2C9 increased ˜3-fold, CYP3A4 increased ˜8-fold, and human 3-hydroxy-3-methylglutaryl-coenzyme A reductase (HMGCR), which is a rate-limiting enzyme in the isoprenoid/cholesterol biosynthesis pathway and involved in cholesterol metabolism (ref. 29; incorporated by reference in its entirety), increased ˜6-fold (FIG. 4B) compared to the sandwich control group. In addition, iPSC-hepatocytes grown within ECM scaffolds had significantly higher mRNA levels compared to less chemically intricate PLLA-collagen scaffolds: CYP1A2 (1.7-fold), CYP2C9 (1.7-fold), and HMGCR (3.3-fold). Synthesis of a mitochondrial membrane respiratory chain enzyme (NDUFA3, FIG. 4C), a glutathione synthesis enzyme (GSTA1, FIG. 4C), and albumin (FIG. 4C) did not show significant differences between ECM scaffolds and either PLLA-collagen scaffolds or sandwich control group.

To investigate potential phenotypic maturation of cells in ECM scaffolds, mRNA transcripts of fetal markers CYP3A7 and AFP in iPSC-hepatocytes grown in ECM scaffolds displayed a decreasing trend at day 14 (CYP3A7, 49% of sandwich control; AFP, 47% of sandwich control,). However, both CYP3A7 and AFP remained essentially unchanged in the PLLA-collagen scaffold (CYP3A7, 93% of sandwich control; AFP 94% of sandwich control, FIG. 4D).

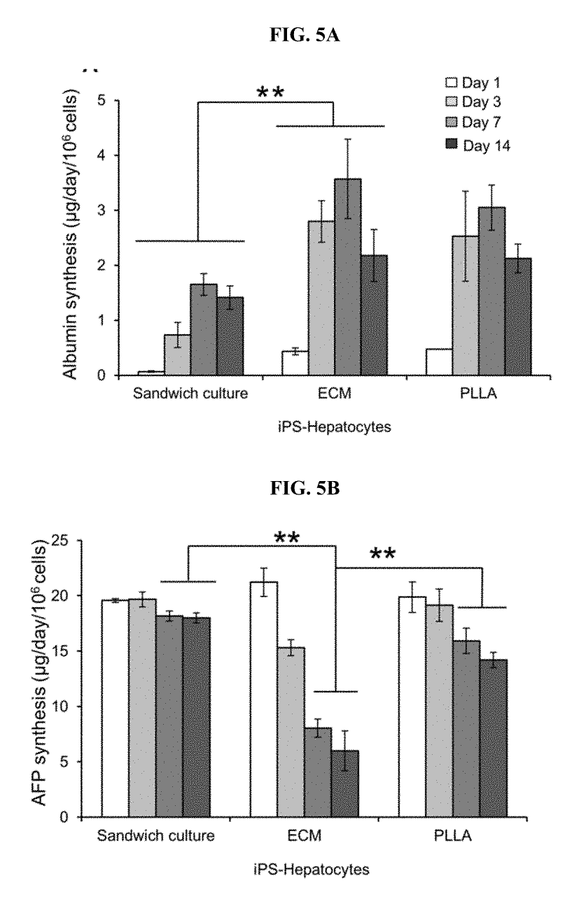

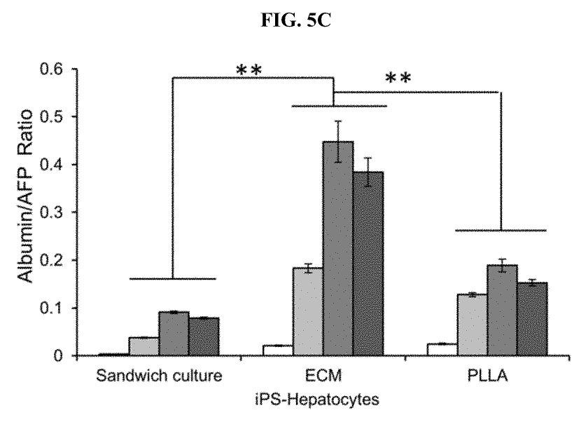

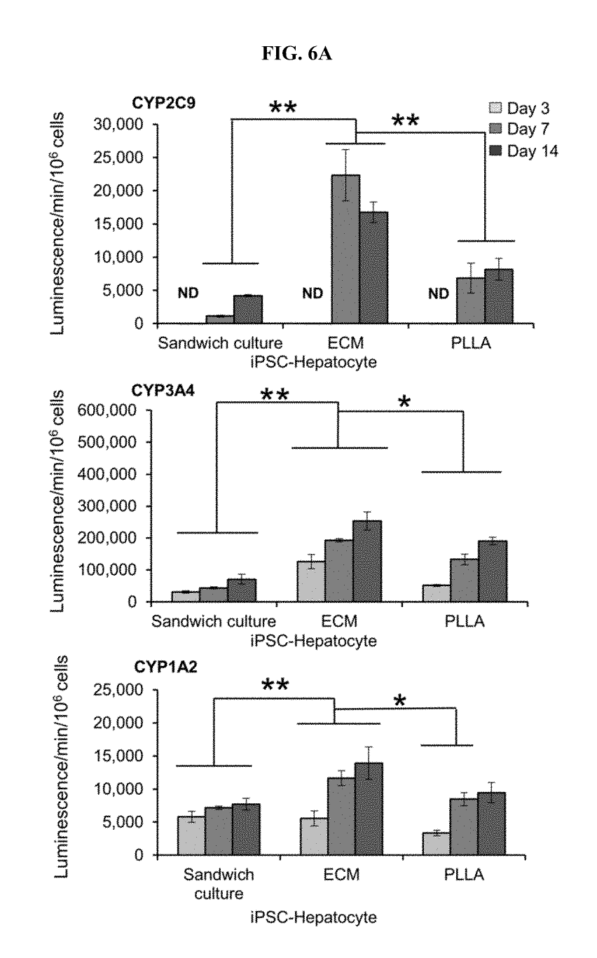

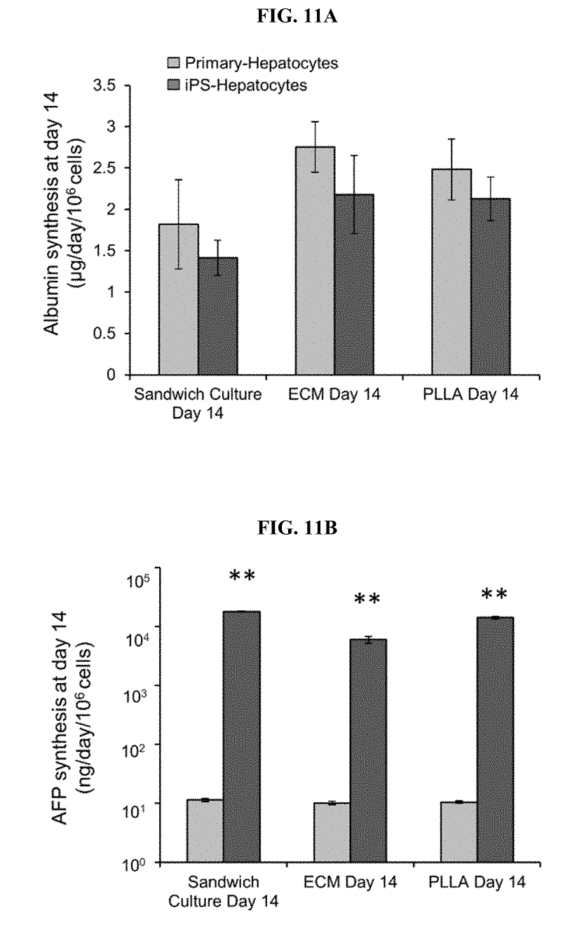

Synthesis of albumin by iPSC-hepatocytes grown within the ECM was significantly higher than the sandwich control group throughout the 14-day culture period (FIG. 5A), and was similar to the PLLA-collagen scaffolds. Nonetheless, at day 14, albumin synthesis by iPSC-hepatocytes grown within ECM scaffolds (2.18±0.47 μg/day/10⁶ cells) was slightly lower than day 7 (FIG. 5A), but not significantly different than primary human hepatocytes grown within ECM scaffolds (2.75±0.31 μg/day/10⁶ cells, FIG. 11) over the same time course.

AFP production in iPSC-hepatocytes grown within ECM scaffolds consistently and significantly decreased from a baseline on day 1 (21.21±1.27 μg/day/10⁶ cells) through day 7 (8.03±0.82 μg/day/10⁶ cells) to day 14 (5.98±1.78 μg/day/10⁶ cells, FIG. 5B). Yet, AFP synthesis in iPSC-hepatocytes grown within the sandwich control environment did not show any significant decline during this interval. Moreover, AFP production in PLLA-collagen scaffolds decreased slightly until day 14 (19.86±1.39 μg/day/10⁶ cells, day 1; 14.19±0.69 μg/day/10⁶ cells, day 14), though did not reach the low level of synthesis found in iPSC-hepatocytes grown in ECM scaffolds, indicating that the more complex biochemical milieu within ECM scaffolds leads to the enhanced maturation of iPSC-hepatocytes. Nonetheless, AFP synthesis in primary human hepatocytes remained low compared to iPSC-hepatocytes grown in all environments (FIG. 11). Using the ratio of albumin to AFP synthesis as a gauge of cellular maturation, iPSC-hepatocytes grown in ECM scaffolds consistently had a higher ratio compared to cells grown in either PLLA-collagen scaffolds or sandwich culture (FIG. 5C), clearly demonstrating cellular maturation in the ECM bioscaffold system.

### Increased Liver-Specific Function in 3D ECM Scaffolds

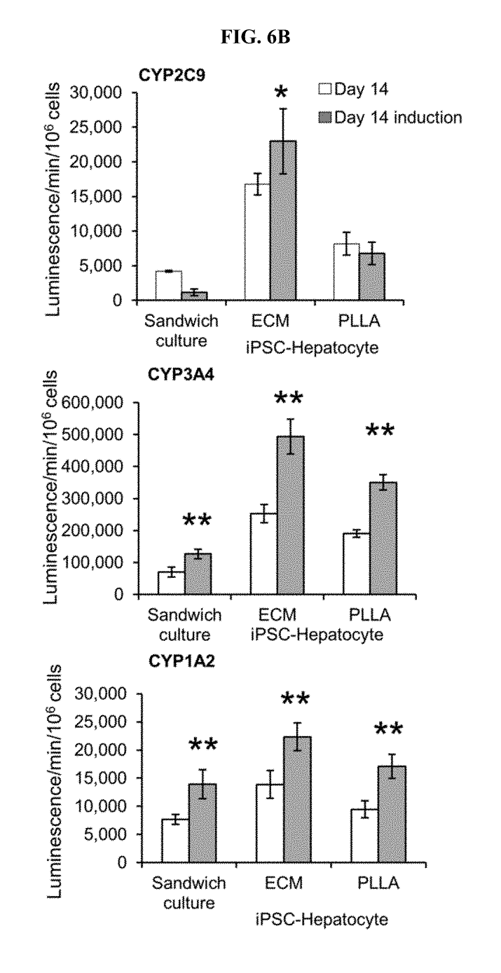

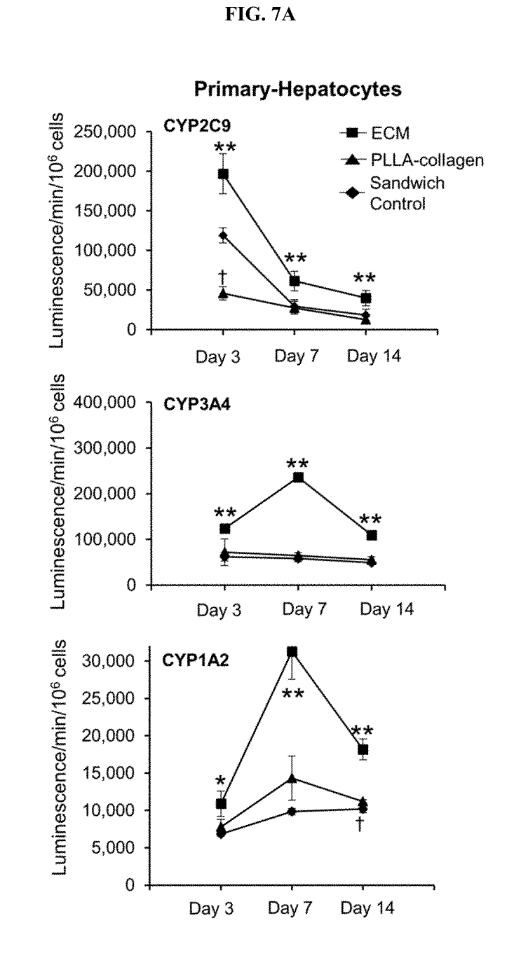

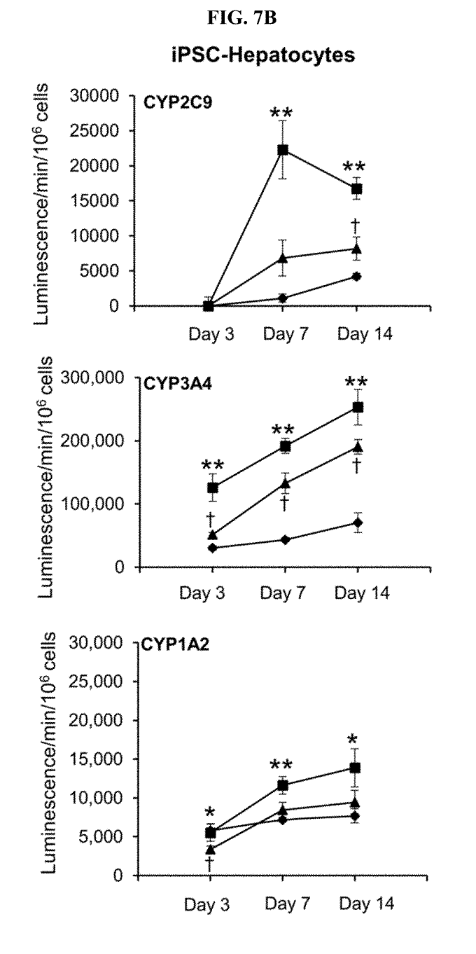

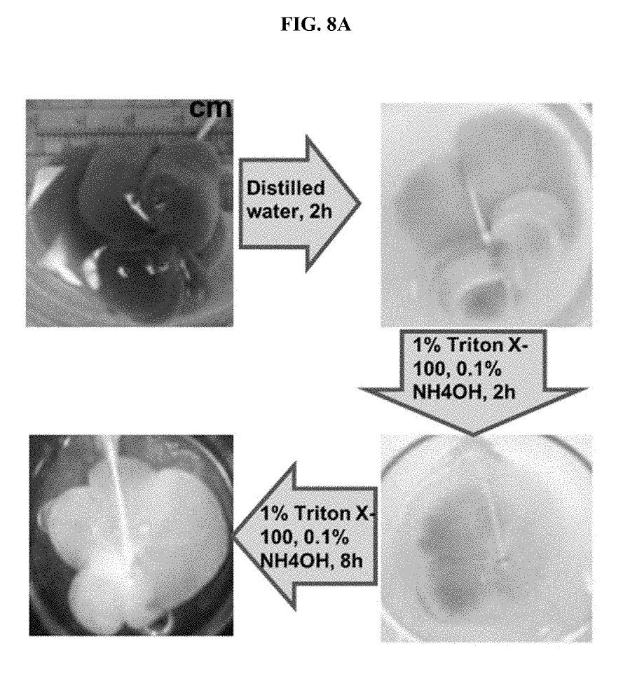

Notably, CYP2C9 activity was nearly undetectable in iPSC-hepatocytes prior to growth within scaffolds (Supplementary FIG. 2C) or when grown within the sandwich control environment, ECM, or PLLA-collagen scaffolds at day 3 (FIG. 6A). Nonetheless, on day 7, CYP2C9 activity of iPSC-hepatocytes in ECM scaffolds increased 20.2-fold (FIG. 6A) beyond that of iPSC-hepatocytes grown in sandwich culture on the same day, and was also significantly higher than iPSC-hepatocytes in PLLA-collagen scaffolds (FIG. 6A). CYP3A4 activity in iPSC-hepatocytes grown within ECM scaffolds at day 14 was 3.6-fold higher than iPSC-hepatocytes in the sandwich control group and 1.3-fold higher than iPSC-hepatocytes in the PLLA-collagen scaffold. CYP1A2 activity of iPSC-hepatocytes grown on ECM scaffolds at day 14 was 1.8-fold higher than iPSC-hepatocytes in the sandwich control group and 1.5-fold higher than iPSC-hepatocytes in PLLA-collagen scaffold (FIG. 6A). Rifampicin induced CYP3A4 activity by 2.0-fold in iPSC-hepatocytes within ECM scaffolds, slightly more than iPSC-hepatocytes within either the sandwich control or PLLA-collagen scaffold on day 14 (FIG. 6B). Yet, induction of CYP1A2 (by 3-methylcholanthrene) or CYP2C9 (by rifampicin) in iPSC-hepatocytes was modest in comparison to primary hepatocytes. Taken together, activity of CYP1A2, CYP2C9 and CYP3A4 in iPSC-hepatocytes tended to increase in 3D culture, with ECM scaffolds conferring the most robust phenotype while primary hepatocytes generally lost activity in all three environments over two weeks (FIG. 7).

All publications and patents mentioned above and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references, some of which are cited above by number, are herein incorporated by reference in their entireties.
1. Cayo M A, Cai J, DeLaForest A et al. JD induced pluripotent stem cell-derived hepatocytes faithfully recapitulate the pathophysiology of familial hypercholesterolemia. Hepatology. 2012; 56:2163-2171.
2. Schwartz R E, Trehan K, Andrus L et al. Modeling hepatitis C virus infection using human induced pluripotent stem cells. Proc Natl Acad Sci USA. 2012; 109:2544-2548.
3. Ebert A D, Yu J, Rose F F, Jr. et al. Induced pluripotent stem cells from a spinal muscular atrophy patient. Nature. 2009; 457:277-280.
4. Ye L, Chang J C, Lin C et al. Induced pluripotent stem cells offer new approach to therapy in thalassemia and sickle cell anemia and option in prenatal diagnosis in genetic diseases. Proc Natl Acad Sci USA. 2009; 106:9826-9830.
5. Raya A, Rodriguez-Piza I, Guenechea G et al. Disease-corrected haematopoietic progenitors from Fanconi anaemia induced pluripotent stem cells. Nature. 2009; 460:53-59.
6. Lee G, Papapetrou E P, Kim H et al. Modelling pathogenesis and treatment of familial dysautonomia using patient-specific iPSCs. Nature. 2009; 461:402-406.
7. Sirenko O, Cromwell E F, Crittenden C et al. Assessment of beating parameters in human induced pluripotent stem cells enables quantitative in vitro screening for cardiotoxicity. Toxicology and Applied Pharmacology. 2013; 273:500-507.
8. Zhu S, Rezvani M, Harbell J et al. Mouse liver repopulation with hepatocytes generated from human fibroblasts. Nature. 2014; 508:93-97.
9. Kamao H, Mandai M, Okamoto S et al. Characterization of human induced pluripotent stem cell-derived retinal pigment epithelium cell sheets aiming for clinical application. Stem Cell Reports. 2014; 2:205-218.
10. Rashid S T, Corbineau S, Hannan N et al. Modeling inherited metabolic disorders of the liver using human induced pluripotent stem cells. J Clin Invest. 2010; 120:3127-3136.
11. Zhang S, Chen S, Li W et al. Rescue of ATP7B function in hepatocyte-like cells from Wilson's disease induced pluripotent stem cells using gene therapy or the chaperone drug curcumin. Hum Mol Genet. 2011; 20:3176-3187.
12. Adams R M, Wang M, Crane A M et al. Effective cryopreservation and long-term storage of primary human hepatocytes with recovery of viability, differentiation, and replicative potential. Cell Transplant. 1995; 4:579-586.
13. Cai J, Zhao Y, Liu Y et al. Directed differentiation of human embryonic stem cells into functional hepatic cells. Hepatology. 2007; 45:1229-1239.
14. Duan Y, Catana A, Meng Y et al. Differentiation and enrichment of hepatocyte-like cells from human embryonic stem cells in vitro and in vivo. Stem Cells. 2007; 25:3058-3068.
15. Hay D C, Fletcher J, Payne C et al. Highly efficient differentiation of hESCs to functional hepatic endoderm requires ActivinA and Wnt3a signaling. Proc Natl Acad Sci USA. 2008; 105:12301-12306.
16. Basma H, Soto-Gutierrez A, Yannam G R et al. Differentiation and transplantation of human embryonic stem cell-derived hepatocytes. Gastroenterology. 2009; 136:990-999.
17. Si-Tayeb K, Noto F K, Nagaoka M et al. Highly efficient generation of human hepatocyte-like cells from induced pluripotent stem cells. Hepatology. 2010; 51:297-305.
18. Kubo A, Kim Y H, Irion S et al. The homeobox gene Hex regulates hepatocyte differentiation from embryonic stem cell-derived endoderm. Hepatology. 2010; 51:633-641.
19. Sullivan G J, Hay D C, Park I H et al. Generation of functional human hepatic endoderm from human induced pluripotent stem cells. Hepatology. 2010; 51:329-335.
20. Sancho-Bru P, Roelandt P, Narain N et al. Directed differentiation of murine-induced pluripotent stem cells to functional hepatocyte-like cells. J Hepatol. 2011; 54:98-107.
21. Berger D R, Ware B R, Davidson M D et al. Enhancing the functional maturity of iPSC-derived human hepatocytes via controlled presentation of cell-cell interactions in vitro. Hepatology. 2014.
22. Gieseck R L, 3rd, Hannan N R, Bort R et al. Maturation of induced pluripotent stem cell derived hepatocytes by 3D-culture. PLoS One. 2014; 9:e86372.
23. Takayama K, Kawabata K, Nagamoto Y et al. 3D spheroid culture of hESC/hiPSC-derived hepatocyte-like cells for drug toxicity testing. Biomaterials. 2013; 34:1781-1789.
24. Loneker A E, Faulk D M, Hussey G S et al. Solubilized liver extracellular matrix maintains primary rat hepatocyte phenotype in-vitro. Journal of Biomedical Materials Research Part A. 2015.
25. Soto-Gutierrez A, Zhang L, Medberry C et al. A whole-organ regenerative medicine approach for liver replacement. Tissue Engineering Part C, Methods. 2011; 17:677-686.
26. Baptista P M, Siddiqui M M, Lozier G et al. The use of whole organ decellularization for the generation of a vascularized liver organoid. Hepatology. 2011.
27. Kajbafzadeh A M, Javan-Farazmand N, Monajemzadeh M et al. Determining the optimal decellularization and sterilization protocol for preparing a tissue scaffold of a human-sized liver tissue. Tissue Engineering Part C Methods. 2013; 19:642-651.
28. Wertheim J A, Baptista P M, Soto-Gutierrez A. Cellular therapy and bioartificial approaches to liver replacement. Curr Opin Organ Transplant. 2012; 17:235-240.
29. Goldstein J L, Brown M S. Regulation of the mevalonate pathway. Nature. 1990; 343:425-430.
30. Crapo P M, Gilbert T W, Badylak S F. An overview of tissue and whole organ decellularization processes. Biomaterials. 2011; 32:3233-3243.
31. Song Z, Cai J, Liu Y et al. Efficient generation of hepatocyte-like cells from human induced pluripotent stem cells. Cell Research. 2009; 19:1233-1242.
32. Wang Y, Cui C B, Yamauchi M et al. Lineage restriction of human hepatic stem cells to mature fates is made efficient by tissue-specific biomatrix scaffolds. Hepatology. 2011; 53:293-305.
33. Nash R A, Hutton G J, Racke M K et al. High-Dose Immunosuppressive Therapy and Autologous Hematopoietic Cell Transplantation for Relapsing-Remitting Multiple Sclerosis (HALT-MS): A 3-Year Interim Report. JAMA Neurol. 2015; 72:159-169.
34. Nagaoka M, Kobayashi M, Kawai C et al. Design of a Vitronectin-Based Recombinant Protein as a Defined Substrate for Differentiation of Human Pluripotent Stem Cells into Hepatocyte-Like Cells. PLoS One. 2015; 10:e0136350.
35. Jakus A E, Rutz A L, Shah R N. Advancing the field of 3D biomaterial printing. Biomed Mater. 2016; 11:014102.
36. Rutz A L, Hyland K E, Jakus A E et al. A multimaterial bioink method for 3D printing tunable, cell-compatible hydrogels. Adv Mater. 2015; 27:1607-1614.
37. Soto-Gutierrez A, Zhang L, Medberry C et al. A whole-organ regenerative medicine approach for liver replacement. Tissue Engineering Part C, Methods. 2011; 17:677-686.

1. A system, comprising:
(a) a bioscaffold comprising acellular hepatic extracellular matrix; and
(b) induced pluripotent stem cell (iPSC)-derived hepatocytes.

2. The system of claim 1, further comprising hepatocyte media.

3. The system of claim 1, wherein the bioscaffold displays one or more peptide, nucleic acid, or small molecule agents.

4. The system of claim 1, further comprising one or more additional cell types.

5. The system of claim 4, comprising stromal cells.

6. A cell population comprising hepatocyte-like cells produced by the system of claim 1.

7. A method of improving liver function in a subject comprising transplanting the cell population of claim 6 into the subject.

8. The method of claim 7, wherein the iPSCs are derived from somatic cells from the subject.

9. The method of claim 7, wherein the iPSCs are derived from somatic cells from a source other than the subject.

10. A liver assist device comprising the cell population of claim 6 into the subject.

11. A screening method comprising exposing the cell population of claim 6 to a test agent, and monitoring the effect on the cell population.

12. A method of generating mature induced pluripotent stem cell (iPSC)-derived hepatocytes, comprising:
(a) providing a bioscaffold derived from a decellularized liver tissue and comprising acellular hepatic extracellular matrix (ECM);
(b) seeding the bioscaffold with iPSC-derived hepatocytes; and
(c) culturing the iPSC-derived hepatocytes within the bioscaffold under conditions that facilitate maturation of hepatocytes.

13. The method of claim 12, wherein conditions that facilitate maturation of hepatocytes comprise substantially atmospheric pressure, about 37° C., and in hepatocyte media.

14. A cell population comprising hepatocytes produced by the method of claim 12.

15. A method of improving liver function in a subject comprising transplanting the cell population of one of claim 14 into the subject.

16. The method of claim 15, wherein the iPSCs are derived from somatic cells from the subject.

17. The method of claim 15, wherein the iPSCs are derived from somatic cells from a source other than the subject.

18. A liver assist device comprising the cell population of claim 14 into the subject.

19. A screening method comprising exposing the cell population of claim 14 to a test agent, and monitoring the effect on the cell population.


SEQUENCE LISTING (20 sequences)

SEQ ID NO: 1 - DNA, 22 bp, Artificial sequence
  Synthetic Primer
cacaggaggaaagagcatctac

SEQ ID NO: 2 - DNA, 21 bp, Artificial sequence
  Synthetic Primer
cacagacggtcactcaaagaa

SEQ ID NO: 3 - DNA, 21 bp, Artificial sequence
  Synthetic Primer
cctgttggagtggcaggaccc

SEQ ID NO: 4 - DNA, 22 bp, Artificial sequence
  Synthetic Primer
tggtgctggccacaagacaacc

SEQ ID NO: 5 - DNA, 21 bp, Artificial sequence
  Synthetic Primer
agcatgggcagtagctcgcct

SEQ ID NO: 6 - DNA, 21 bp, Artificial sequence
  Synthetic Primer
aggtccgccctgtcatcagca

SEQ ID NO: 7 - DNA, 20 bp, Artificial sequence
  Synthetic Primer
ggatttgcctctgtgccgcc

SEQ ID NO: 8 - DNA, 21 bp, Artificial sequence
  Synthetic Primer
gcagccaggccatctgctctt

SEQ ID NO: 9 - DNA, 23 bp, Artificial sequence
  Synthetic Primer
tcccagtctgttcccttctcggc

SEQ ID NO: 10 - DNA, 20 bp, Artificial sequence
  Synthetic Primer
ttgacagtgccaggtgcggg

SEQ ID NO: 11 - DNA, 22 bp, Artificial sequence
  Synthetic Primer
gtttctacagcctggcagccca

SEQ ID NO: 12 - DNA, 22 bp, Artificial sequence
  Synthetic Primer
agttcttggcctccatgactgc

SEQ ID NO: 13 - DNA, 20 bp, Artificial sequence
  Synthetic Primer
cgtgtccttcgtcgtcgggg

SEQ ID NO: 14 - DNA, 20 bp, Artificial sequence
  Synthetic Primer
ctgggcacgtcgggcatgtt

SEQ ID NO: 15 - DNA, 21 bp, Artificial sequence
  Synthetic Primer
gctgacctggctaccatattt

SEQ ID NO: 16 - DNA, 21 bp, Artificial sequence
  Synthetic Primer
gggatgccttcttgctatctc

SEQ ID NO: 17 - DNA, 22 bp, Artificial sequence
  Synthetic Primer
ctgagaagttcctccctgaaag

SEQ ID NO: 18 - DNA, 22 bp, Artificial sequence
  Synthetic Primer
gcacgtacagaatccctgatta

SEQ ID NO: 19 - DNA, 21 bp, Artificial sequence
  Synthetic Primer
ctgcttctcacgggactattt

SEQ ID NO: 20 - DNA, 22 bp, Artificial sequence
  Synthetic Primer
cctcccaaactgctaggattac